(19) United States
(12) Patent Application Publication
(10) Pub. No.: US 2007/0196423 A1
(43) Pub. Date: Aug. 23, 2007

(54) Implantable medical device coatings with biodegradable elastomer and releasable therapeutic agent

(75) Inventors: Patrick H. Ruane, Redwood City, CA (US); Waleska Perez-Segarra, West Lafayette, IN (US); Priscilla Reyes, West Lafayette, IN (US)

Correspondence Address: BRINKS HOFER GILSON & LIONE/INDY/COOK, ONE INDIANA SQUARE, SUITE 1600, INDIANAPOLIS, IN, 46204-2033, US

(73) Assignee: MED Institute, Inc., West Lafayette, IN (US)

(21) Appl. No.: 11/599,850

(22) Filed: Nov. 15, 2006

Related U.S. Application Data
(60) Provisional application No. 60/738,475, filed on Nov. 21, 2005.
(60) Provisional application No. 60/738,476, filed on Nov. 21, 2005.
(60) Provisional application No. 60/830,660, filed on Jul. 13, 2006.

(51) Int. Cl. A61L 33/00

(52) U.S. Cl. 424/423; 427/2.25

(57) ABSTRACT

A coated medical device, such as a stent, that elutes a therapeutic agent in a controlled manner is provided. The medical device may be coated with a layer of therapeutic agent and a layer of bioabsorbable elastomer over the layer of therapeutic agent. Methods of manufacturing a coated medical device and of coating a medical device are also provided.

### RELATED APPLICATIONS

This application claims the benefit of U.S. Provisional Patent Application Ser. Nos. 60/738,475, filed Nov. 21, 2005; 60/738,476, filed Nov. 21, 2005; and 60/830,660, filed Jul. 13, 2006, all of which are incorporated by reference herein in their entirety.

### TECHNICAL FIELD

The present invention relates to implantable medical device coatings configured to release a therapeutic agent. More specifically, the present invention relates to implantable medical device coatings comprising a biodegradable elastomer and a therapeutic agent, as well as related methods of coating the implantable medical device, and methods for the local administration of the therapeutic agents to a target site in a body vessel.

### BACKGROUND

Delivery of a therapeutic agent from an implantable medical device can be desirable for a variety of applications. Therapeutic agents can be released from a medical device, such as an expandable stent or valve, to treat or mitigate undesirable conditions including restenosis, tumor formation or thrombosis. Procedures for mitigating certain conditions can include implantation of a device comprising a therapeutic agent. For example, the implantation of stents during angioplasty procedures has substantially advanced the treatment of occluded body vessels. Angioplasty procedures such as Percutaneous Transluminal Coronary Angioplasty (PCTA) can widen a narrowing or occlusion of a blood vessel by dilation with a balloon. Occasionally, angioplasty may be followed by an abrupt closure of the vessel or by a more gradual closure of the vessel, commonly known as restenosis. Acute closure may result from an elastic rebound of the vessel wall and/or by the deposition of blood platelets and fibrin along a damaged length of the newly opened blood vessel. In addition, restenosis may result from the natural healing reaction to the injury to the vessel wall (known as intimal hyperplasia), which can involve the migration and proliferation of medial smooth muscle cells that continues until the vessel is again occluded. To prevent such vessel occlusion, stents have been implanted within a body vessel. However, restenosis may still occur over the length of the stent and/or past the ends of the stent where the inward forces of the stenosis are unopposed. To reduce this problem, one or more therapeutic agents may be administered to the patient. For example, a therapeutic agent may be administered systemically, locally administered through a catheter positioned within the body vessel near the stent, or coated on the stent itself.

A medical device can be coated with a therapeutic agent in a manner suitable to expose tissue near the implantation site of the medical device to the therapeutic agent over a desired time interval, such as by releasing the therapeutic agent from an implanted stent into surrounding tissue inside a body vessel. Various approaches can be used to control the rate and dose of release of therapeutic agents from an implantable medical device. The design configuration of an implantable device can be adapted to influence the release of therapeutic from the device. A therapeutic agent can be included in the implantable medical device in various configurations. In some devices, the therapeutic agent is contained within an implantable frame or within a coating on the surface of the implantable frame. An implantable frame coating can include a bioabsorbable material mixed with a therapeutic agent, or coated over the therapeutic agent. Some implantable medical devices comprise an implantable frame with a bioabsorbable material mixed with or coated over a therapeutic agent. For example, U.S. Pat. No. 5,624,411 to Tuch, filed Jun. 7, 1995, describes radially expandable stents coated with a porous polymer overlaying a first coating layer containing various bioactive agents. The porous polymer may be a biodegradable polymer, such as poly(lactic acid). Implantable medical devices can also comprise a porous biostable material containing a dissolvable material and a therapeutic agent, where dissolution of the removeable material upon implantation forms pores that release the therapeutic agent. For example U.S. Pat. No. 5,447,724 to Helmus, filed Nov. 15, 1993, describes a two-layer coating comprising an outer layer containing a mixture of a biostable polymer and an elutable component positioned over a bioactive reservoir layer such that the elutable component dissolves away upon implantation of the coating in a body, transforming the outer layer into a porous layer permitting diffusion of the bioactive agent from the reservoir layer through the outer layer and into the body.

The design of a controlled release medical device can also depend on the desired mode of implantation of the device. The device can be adapted to the appropriate biological environment in which it is used. For example, a device for percutaneous transcatheter implantation can be sized and configured for implantation from the distal portion of a catheter, adapted for expansion at the point of treatment within the body vessel by balloon or self-expansion. An implantable medical device can also be adapted to withstand a desired amount of flexion or impact, and should provide delivery of a therapeutic agent with a desired elution rate for a desired period of time.

There is a need for a medical device capable of releasing a therapeutic agent at a desired rate and over a desired time period upon implantation. Preferably, implantation of a medical device releases a therapeutic agent as needed at the site of medical intervention to promote a therapeutically desirable outcome, such as mitigation of restenosis. There is also a need for such a medical device with a releasable therapeutic agent capable of withstanding the flexion and impact that accompany the transportation and implantation of the device without releasing an undesirable amount of the therapeutic agent prior to implantation at a point of treatment. For example, a medical device can include a coating of a bioabsorbable material with sufficient durability to resist the undesirable premature release of the therapeutic agent from the device prior to implantation at a point of treatment within a body vessel.

### SUMMARY

The present invention relates to implantable medical device coatings configured to release a therapeutic agent. The implantable medical device preferably includes a multi-layer coating that releases a hydrophobic therapeutic agent upon implantation in a body vessel. The coating preferably includes at least two layers, with a layer of a bioabsorbable elastomer positioned over a layer comprising the therapeutic agent.

In a first embodiment, durable implantable medical device coatings are provided that release a therapeutic agent over a desired period of time. The coatings preferably include a layer of a biodegradable elastomer positioned over a layer of a hydrophobic therapeutic agent. A two-layer coating may be formed from a first layer comprising a taxane therapeutic agent coated with a second layer comprising a poly(lactic acid) polymer. The first layer may be formed from a therapeutically effective amount of a suitable therapeutic agent, such as paclitaxel, although the first layer may include any suitable hydrophobic therapeutic agent(s). For example, the first layer may comprise, or consist essentially of, 0.05 to 1.00 μg of paclitaxel per mm² of the first layer on the abluminal surface. Preferably, the coated medical device contains a total of less than 1.00 μg of paclitaxel per mm² of the coated surface. The first layer is preferably enclosed by portions of the second layer and the vascular stent so that the first layer does not form any portion of the outer surface of the coated medical device before contacting the coated vascular stent with an elution medium. In addition, the first layer is preferably substantially polymer-free, containing less than 0.10 μg of the biodegradable elastomer per mm² of the first layer. The second layer may be formed from about 0.05 to 20.00 μg of the biodegradable elastomer, such as poly(lactic acid), per mm² of the second layer on the first layer. Preferably, the second layer comprises or consists essentially of an amorphous poly(lactic acid) selected from the group consisting of: poly(D-lactic acid), poly(L-lactic acid) and poly(D,L-lactic acid). Typically, the weight of the biodegradable elastomer in the second layer is 1-20-times greater than the weight of the therapeutic agent in the first layer, depending on the desired elution rate of the therapeutic agent. In addition, the second layer is preferably substantially free of the therapeutic agent, containing less than 0.10 μg of the biodegradable elastomer per mm² of the second layer. Increasing the amount of the biodegradable elastomer in the second layer reduces the rate of elution of the therapeutic agent in an elution medium.

The hydrophobic therapeutic agent can be released from the coating at different rates in an elution medium by altering the ratio of the therapeutic agent and the elastomer. For example, increasing the weight ratio of the biodegradable elastomer in the second layer relative to the weight of the therapeutic agent in the first layer slows the elution of the therapeutic agent. The elution rate may be measured by contacting the coated medical device with an elution medium and measuring the amount and rate of release of the therapeutic agent into the elution medium. Medical device coatings may be characterized by measuring an elution profile, which records the rate of elution of the therapeutic agent from the coating into the elution medium as a function of time. The shape and characteristics elution profile of a medical device coating depends on the elution medium chosen. Examples of suitable elution media that typically provide different elution profiles include porcine serum, aqueous solutions comprising a cyclodextrin, phosphate buffered serum (PBS), bovine serum albumin (BSA), sodium dodecyl sulfate (SDS), ethanol and blood. In one aspect, medical device coatings may be characterized by the elution profile of the therapeutic agent into a porcine serum elution medium for 24 hours in a porcine serum elution assay, wherein the coating is contacted with a porcine serum elution medium prepared by adding 0.104 mL of a 6.0 g/L Heparin solution to porcine serum at 37° C. and adjusting the pH to 5.6+/−0.3 using a 20% v/v aqueous solution of acetic acid at a flow rate of 16 mL/minute. In another aspect, the coating may be characterized by measuring different elution profile in an aqueous solution containing 0.1% and 10% by volume of a cyclodextrin. Preferably, the cyclodextrin is a 0.5% aqueous solution of Heptakis-(2,6-di-O-methyl)-β-cyclodextrin at 25° C. Using an elution medium comprising a cyclodextrin typically provides more rapid elution of a hydrophobic therapeutic agent, such as paclitaxel, providing a shorter time period for measuring the relative elution rates of different coating configurations.

In a second aspect of the first embodiment, multi-layer drug-eluting coatings with improved durability are provided. In particular, the durability of coatings comprising a biodegradable elastomer can be improved by selecting a biodegradable elastomer of a preferred molecular weight of less than about 250,000 kDa, and preferably a molecular weight of about 75,000 kDa to 250,000 kDa. The coating durability is preferably characterized by a weight loss of less than 10%, more preferably less than 5%, of the coating weight during sterilization and packaging. For example, for coated radially-expandable vascular stents, a coating weight loss of 5% or less may be achieved during the steps of crimping the coated vascular stent onto a delivery catheter, sterilizing of the coated vascular stent by standard ethylene oxide sterilization methods and subsequent deployment of the stent by radial expansion.

The coated implantable medical device is preferably configured as a radially-expandable cylindrical vascular stent having an abluminal (exterior) surface and a luminal surface defining a substantially tubular lumen extending axially through the stent. The vascular stent may include a plurality of openings between the abluminal and luminal surfaces. Preferably, the coating is applied to the abluminal surface. More preferably, the coating is not applied to the luminal surface. The coated implantable medical device coating may be configured to release a therapeutic agent adhered to a surface of the medical device over a desired period of time. Preferably, the coating comprises or consists of two layers: a first layer comprising a therapeutically effective amount of a therapeutic agent positioned between the surface and a second layer comprising a biodegradable elastomer. A second layer positioned over the first layer may comprise a poly(lactic acid) biodegradable elastomer in an amount between 1 and 20 times the weight of the therapeutic agent in the first layer, as described above. Alternatively, the implantable medical device may be configured as any suitable device, including a catheter, a stent graft and a vascular wrap. The coating may be applied to any suitable surface, but is preferably positioned on a surface shaped and configured to contact the wall of a body vessel upon implantation.

In a second embodiment, methods of coating implantable medical devices with a releasable therapeutic agent are provided. Preferably, the methods for coating an implantable medical device to form a drug delivery system, the method include the steps of: (a) providing an implantable medical device having a surface; (b) depositing a first layer consisting essentially of a hydrophobic therapeutic agent on the surface of the medical device by the steps of: applying to the surface a first solution comprising a first solvent and a hydrophobic therapeutic agent dispersed in the first solvent, where the first solution does not contain a polymer; evaporating the first solvent to form the first coating layer consisting essentially of the therapeutic agent on the surface; and repeating the application and evaporation steps until the first layer contains a therapeutically effective amount of a hydrophobic therapeutic agent per mm² of the surface; and (c) depositing a second layer comprising a biodegradable elastomer over the first coating layer on the medical device to form a coated medical device by the steps of: applying to the first layer a second solution comprising a second solvent and a biodegradable elastomer polymer dispersed in the second solvent, the biodegradable elastomer having a molecular weight of 75,000 to 240,000 kDa; evaporating the second solvent to form at least a portion of the second coating layer; and repeating the application and evaporation steps until the weight of the biodegradable elastomer in the second layer is between 1 and 20 times greater than the weight of the therapeutic agent in the first layer.

In a third embodiment, methods of treatment are provided that include the intraluminal placement of a coated implantable medical device within a body vessel. The coated implantable medical device is preferably delivered using a catheter-based delivery system. In one preferred aspect, methods of delivering a therapeutic agent to peripheral blood vessel preferably include the steps of: providing a coated vascular stent described with respect to the first embodiment, intralumenally inserting the coated vascular stent into the blood vascular system using a means for intralumenal delivery comprising a catheter, positioning the coated vascular stent within a peripheral artery and radially expanding the coated vascular stent within the peripheral artery so as to place the coated vascular stent in contact with a portion of a wall of the peripheral artery in a manner effective to deliver the therapeutic agent to the wall of the peripheral artery.

### BRIEF DESCRIPTION OF THE DRAWINGS

FIG. 1A shows side view of an implantable medical device configured as a coated vascular stent.

FIG. 1B shows a cross sectional view of a portion of the coated vascular stent of FIG. 1A.

FIG. 1C shows a cross sectional view of a portion of a first alternative coating configuration for the coated vascular stent of FIG. 1A.

FIG. 2A shows a cross sectional view of a portion of a second alternative device configuration for the coated vascular stent of FIG. 1A.

FIG. 2B shows a cross sectional view of a portion of a third alternative coating configuration for the coated vascular stent of FIG. 1A.

FIG. 3 shows a UV-Visible Spectra for paclitaxel in ethanol.

FIG. 4A shows the paclitaxel elution profile for a medical device without a bioabsorbable elastomer layer.

FIG. 4B shows the paclitaxel elution profile for a medical device with a bioabsorbable elastomer layer.

FIG. 5A shows elution profiles for various medical device coatings in procine serum.

FIG. 5B shows elution profiles for various medical device coatings in procine serum.

FIG. 5C shows an elution profiles of various medical device coatings in porcine serum.

FIG. 5D shows two elution profiles of a two-layer coated medical device comprising a layer of paclitaxel covered by a layer of poly(lactic acid) (PLA). The first elution profile was obtained in a 5% aqueous solution of Heptakis-(2,6-di-O-methyl)-β-cyclodextrin (HCD), and the second elution profile was obtained in porcine serum.

FIG. 5E shows three elution profiles of a two-layer coated medical device comprising a layer of paclitaxel covered by a second layer comprising different amounts of poly(lactic acid) (PLA). Each elution profile was obtained in a 5% aqueous solution of Heptakis-(2,6-di-O-methyl)-β-cyclodextrin (HCD).

FIG. 6A and FIG. 6B are optical micrographs of PLA coatings deposited by ESD using different solvents. FIG. 6C, FIG. 6D and FIG. 6E are Scanning Electron Microscope (SEM) images of certain bioabsorbable coatings deposited by different methods.

FIG. 7A, FIG. 7B, FIG. 7C, and FIG. 7D are SEM images of various PLA coatings.

FIG. 8A, FIG. 8B, FIG. 8C, and FIG. 8D are SEM images of various PLA coatings comprising PLA polymers with different molecular weights.

FIG. 8E and FIG. 8F are elution profile graphs showing the elution of PLA-paclitaxel coatings using PLA coatings with different molecular weights.

FIG. 9 is an angiogram of a porcine iliac and femoral artery after implantation of coated stents therein.

### DETAILED DESCRIPTION

The following detailed description and appended drawings describe and illustrate various exemplary embodiments of the invention. The description and drawings serve to enable one skilled in the art to make and use the invention. Discussion of the illustrated coating configurations of certain preferred coated medical device system comprising a two-layer coating on a vascular stent also relate to other coated medical devices comprising different implantable medical devices (including catheters, stent grafts, vascular grafts, and others) coated with more than two layers, different hydrophobic therapeutic agents, different biodegradable elastomers and/or different layer compositions are also.

Unless otherwise defined, all technical and scientific terms used herein have the same meaning as commonly understood by one of ordinary skill in the art to which this invention pertains. In case of conflict, the present document, including definitions, will control. Preferred methods and materials are described below, although methods and materials similar or equivalent to those described herein can be used in the practice or testing of the present invention.

The term “hydrophobic,” as used herein, refers to a substance with a solubility in water of less than 0.1 mg/mL at room temperature (about 25° C.).

A therapeutic agent is “enclosed” if the therapeutic agent is surrounded by the coating or other portions of the medical device, and does not form a portion of the surface area of the medical device prior to release of the therapeutic agent. When a medical device is initially placed in an elution medium, an enclosed therapeutic agent is preferably not initially in contact with the elution medium.

The term “elution,” as used herein, refers to removal of a material from a medical device coating upon contact with an elution medium. The elution medium can remove the material from the substrate by any process, including by acting as a solvent with respect to the removable material. For example, in medical devices adapted for introduction to the vascular system, blood can act as an elution medium that dissolves a therapeutic agent releasably associated with a portion of the surface of the medical device. The removable material preferably includes the therapeutic agent, but can also include a bioabsorbable elastomer. The elution profile of a given coating configuration and composition typically varies in different elution media.

An “elution medium,” as used herein, refers to a condition or environment into which a therapeutic agent can be released from a coating upon contact of the coating with the elution medium. The elution medium is desirably a fluid. More desirably, the elution medium is a biological fluid such as blood or porcine serum, although any other chemical substance can be used as an elution medium. For example, alternative elution media include phosphate buffered saline, aqueous solutions, reaction conditions including temperature and/or pH, or combinations thereof, that release the therapeutic agent at a desired rate. Preferably, the elution medium is a fluid that provides an elution profile that is similar to the elution profile obtained upon implantation of the medical device within a body vessel. For example, porcine serum can provide an elution profile that is similar to the elution profile in blood for some coating configurations.

The term “effective amount” refers to an amount of an active ingredient sufficient to achieve a desired affect without causing an undesirable side effect. In some cases, it may be necessary to achieve a balance between obtaining a desired effect and limiting the severity of an undesired effect. It will be appreciated that the amount of active ingredient used will vary depending upon the type of active ingredient and the intended use of the composition of the present invention.

The terms “about” or “substantially” used with reference to a quantity includes variations in the recited quantity that are equivalent to the quantity recited, such as an amount that is insubstantially different from a recited quantity for an intended purpose or function.

The term “luminal surface,” as used herein, refers to the portion of the surface area of a medical device defining at least a portion of an interior lumen. Conversely, the term “abluminal surface,” as used herein, refers to portions of the surface area of a medical device that do not define at least a portion of an interior lumen. For example, where the medical device is a tubular frame formed from a plurality of interconnected struts and bends defining a cylindrical lumen, the abluminal surface includes the exterior surface, sides and edges of the struts and bends, while the luminal surface can include the interior surface of the struts and bends.

The term “interface,” as used herein, refers to a common boundary between two structural elements, such as two coating layers in contact with each other.

The term “coating,” as used herein and unless otherwise indicated, refers generally to material attached to an implantable medical device. A coating can include material covering any portion of a medical device, and can be configured with one or more coating layers. A coating can have a substantially constant or a varied thickness and composition. Coatings can be adhered to any portion of a medical device surface, including the luminal surface, the abluminal surface, or any portions or combinations thereof.

The term “coating layer,” as used herein, refers to a material positioned over a substrate surface. A coating layer material can be positioned in contact with the substrate surface, or in contact with other material(s) between the substrate surface and the coating layer material. A coating layer can cover any portion of the surface of a substrate, including material positioned in separate discrete portions of the substrate or a continuous layer over an entire substrate surface.

The term “implantable” refers to an ability of a medical device to be positioned at a location within a body, such as within a body vessel. Furthermore, the terms “implantation” and “implanted” refer to the positioning of a medical device at a location within a body, such as within a body vessel.

The term “alloy” refers to a substance composed of two or more metals or of a metal and a nonmetal intimately united, such as by chemical or physical interaction. Alloys can be formed by various methods, including being fused together and dissolving in each other when molten, although molten processing is not a requirement for a material to be within the scope of the term “alloy.” As understood in the art, an alloy will typically have physical or chemical properties that are different from its components.

The term “mixture” refers to a combination of two or more substances in which each substance retains its own chemical identity and properties.

The term “bioabsorbable” refers to materials selected to dissipate upon implantation within a body, independent of which mechanisms by which dissipation can occur, such as dissolution, degradation, absorption and excretion. The actual choice of which type of materials to use may readily be made by one of ordinary skill in the art. Such materials are often referred to by different terms in the art, such as “bioresorbable,” “bioabsorbable,” or “biodegradable,” depending upon the mechanism by which the material dissipates. The prefix “bio” indicates that the erosion occurs under physiological conditions, as opposed to other erosion processes, caused for example, by high temperature, strong acids or bases, UV light or weather conditions.

The terms “absorption,” “bioresorption” and “bioabsorption” can be used interchangeably to refer to the ability of the polymer or its degradation products to be removed by biological events, such as by fluid transport away from the site of implantation or by cellular activity (e.g., phagocytosis). The term “bioabsorbable” will generally be used in the following description to encompass resorbable, absorbable, bioresorbable, and biodegradable.

A “biocompatible” material is a material that is compatible with living tissue or a living system by not being toxic or injurious.

A “non-bioabsorbable” or “biostable” material refers to a material, such as a polymer or copolymer, which remains in the body without substantial bioabsorption.

The phrase “controlled release” refers to an alteration of the rate of release of a therapeutic agent from a medical device coating in a given environment. A coating or configuration that alters the rate at which the therapeutic agent is released from a medical device provides for the controlled release of the therapeutic agent. A “sustained release” refers to prolonging the rate or duration of release of a therapeutic agent from a medical device. The rate of a controlled release of a therapeutic agent may be constant or vary with time. A controlled release may be characterized by a drug elution profile, which shows the measured rate at which the therapeutic agent is released from a drug-coated device in a given elution medium as a function of time. A controlled release elution profile may include, for example, an initial burst release associated with the introduction of the medical device into the physiological environment, followed by a more gradual subsequent release.

As used herein, the phrase “therapeutic agent” refers to any implantable pharmaceutically active agent that results in an intended therapeutic effect on the body to treat or prevent conditions or diseases. Therapeutic agents include any suitable biologically-active chemical compounds, biologically derived components such as cells, peptides, antibodies, and polynucleotides, and radiochemical therapeutic agents, such as radioisotopes.

An “anti-proliferative” agent/factor/drug indicates any protein, peptide, chemical or molecule that acts to inhibit cell division. Examples of anti-proliferative agents include microtubule inhibitors such as vinblastine, vincristine, colchicine and paclitaxel, or other agents such as cisplatin.

The term “polypeptide” refers to a polymer of amino acid residues. Both full-length proteins and fragments thereof are encompassed by the definition. The term also includes modifications, such as deletions, additions and substitutions (generally conservative in nature), to native sequence.

The term “polysaccharide” refers to a polymer of monosaccharide residues. Some exemplary polysaccharides include low and high molecular weight heparin and dextran, including derivatives of the same, such as dextran sulfate salts and dextran-metal complexes such as dextran-iron complex.

The term “pharmaceutically acceptable,” as used herein, refers to those compounds of the present invention which are, within the scope of sound medical judgment, suitable for use in contact with the tissues of humans and lower mammals without undue toxicity, irritation, and allergic response, are commensurate with a reasonable benefit/risk ratio, and are effective for their intended use, as well as the zwitterionic salt forms of the compounds of the invention.

When naming substances that can exist in multiple enantiomeric forms, reference to the name of the substance without an enantiomeric designation, such as (d) or (l), refers herein to the genus of substances including the (d) form, the (l) form and the racemic mixture (e.g., d, l), unless otherwise specified. For example, recitation of “poly(lactic acid),” unless otherwise indicated, refers to a compound selected from the group consisting of: poly(L-lactic acid), poly(D-lactic acid) and poly(D,L-lactic acid). Similarly, generic reference to compounds that can exist in two or more polymorphs is understood to refer to the genus consisting of each individual polymorph species and any combinations or mixtures thereof.

As used herein, “derivative” refers to a chemically or biologically modified version of a chemical compound that is structurally similar to a parent compound and (actually or theoretically) derivable from that parent compound. A derivative may or may not have different chemical or physical properties of the parent compound. For example, the derivative may be more hydrophilic or it may have altered reactivity as compared to the parent compound. Derivatization (i.e., modification) may involve substitution of one or more moieties within the molecule (e.g., a change in functional group). For example, a hydrogen may be substituted with a halogen, such as fluorine or chlorine, or a hydroxyl group (—OH) may be replaced with a carboxylic acid moiety (—COOH). The term “derivative” also includes conjugates, and prodrugs of a parent compound (i.e., chemically modified derivatives which can be converted into the original compound under physiological conditions). For example, the prodrug may be an inactive form of an active agent. Under physiological conditions, the prodrug may be converted into the active form of the compound. Prodrugs may be formed, for example, by replacing one or two hydrogen atoms on nitrogen atoms by an acyl group (acyl prodrugs) or a carbamate group (carbamate prodrugs). More detailed information relating to prodrugs is found, for example, in Fleisher et al., Advanced Drug Delivery Reviews 19 (1996) 115; Design of Prodrugs, H. Bundgaard (ed.), Elsevier, 1985; or H. Bundgaard, Drugs of the Future 16 (1991) 443. The term “derivative” is also used to describe all solvates, for example hydrates or adducts (e.g., adducts with alcohols), active metabolites, and salts of the parent compound. The type of salt that may be prepared depends on the nature of the moieties within the compound. For example, acidic groups, for example carboxylic acid groups, can form, for example, alkali metal salts or alkaline earth metal salts (e.g., sodium salts, potassium salts, magnesium salts and calcium salts, and also salts with physiologically tolerable quaternary ammonium ions and acid addition salts with ammonia and physiologically tolerable organic amines such as, for example, triethylamine, ethanolamine or tris- (2-hydroxyethyl)amine). Basic groups can form acid addition salts, for example with inorganic acids such as hydrochloric acid, sulfuric acid or phosphoric acid, or with organic carboxylic acids and sulfonic acids such as acetic acid, citric acid, benzoic acid, maleic acid, fumaric acid, tartaric acid, methanesulfonic acid or p-toluenesulfonic acid. Compounds which simultaneously contain a basic group and an acidic group, for example a carboxyl group in addition to basic nitrogen atoms, can be present as zwitterions. Salts can be obtained by customary methods known to those skilled in the art, for example by combining a compound with an inorganic or organic acid or base in a solvent or diluent, or from other salts by cation exchange or anion exchange.

As used herein, “analogue” refers to a chemical compound that is structurally similar to another but differs slightly in composition (as in the replacement of one atom by an atom of a different element or in the presence of a particular functional group), but may or may not be derivable from the parent compound. A “derivative” differs from an “analogue” in that a parent compound may be the starting material to generate a “derivative,” whereas the parent compound may not necessarily be used as the starting material to generate an “analogue.”

Any concentration ranges, percentage range, or ratio range recited herein are to be understood to include concentrations, percentages or ratios of any integer within that range and fractions thereof, such as one tenth and one hundredth of an integer, unless otherwise indicated. Also, any number range recited herein relating to any physical feature, such as polymer subunits, size or thickness, are to be understood to include any integer within the recited range, unless otherwise indicated. It should be understood that the terms “a” and “an” as used above and elsewhere herein refer to “one or more” of the enumerated components. For example, “a” polymer refers to one polymer or a mixture comprising two or more polymers.

Coating Configurations

In a first embodiment, the implantable medical device includes a multi-layer coating that releases a therapeutic agent upon implantation in a body vessel. Preferably, the coating includes at least two layers: a first layer comprising a hydrophobic therapeutic agent positioned between at least a portion of the abluminal surface of the medical device and a second layer comprising a bioabsorbable elastomer positioned over and covering the first layer.

The medical device is preferably configured to position the second layer between the first layer and the wall of a body vessel upon implantation. Preferably, the medical device structure defines an interior lumen with a luminal (interior) surface positioned radially opposite an abluminal (exterior) surface. The coating can be applied to the luminal and/or the abluminal surface. Preferably, a therapeutic agent is releasibly attached to the abluminal surface. Optionally, a second therapeutic agent can be releasibly attached to the luminal surface. One or more bioabsorbable elastomers can cover the therapeutic agent(s). The bioabsorbable elastomer preferably encloses the therapeutic agent(s), and can be applied to the abluminal surface as well as the luminal surface of the medical device. Each coating layer can be applied to a portion of a surface or can be applied continuously over the entire surface, depending on the device configuration desired.

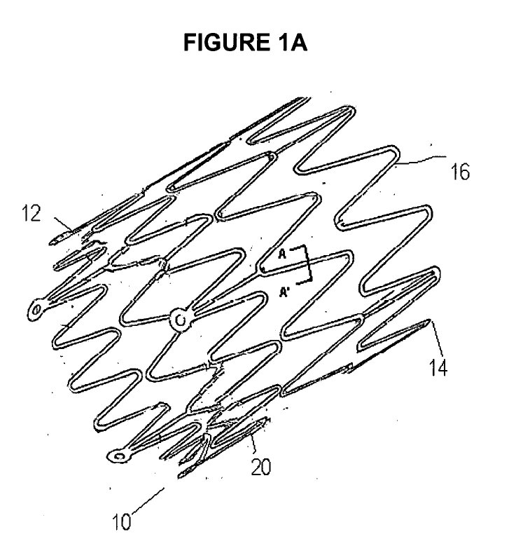

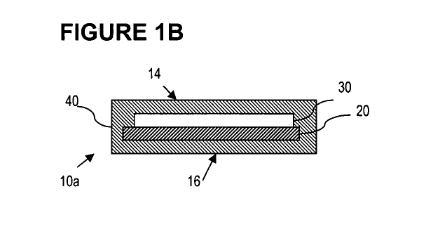

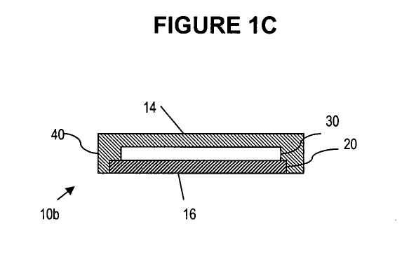

FIG. 1 shows an exemplary coated medical device configured as a coated implantable vascular stent 10 having a two layer coating over a radially expandable frame 20. The vascular stent 10 can be a tubular stent formed from a plurality of connected hoops 12 formed from a sinusoidal array of alternating struts and bends. The vascular stent 10 can be radially expandable from compressed state to the expanded state shown in FIG. 1. The frame 20 can be formed from any suitable material, such as a superelastic nickel-titanium alloy.

The abluminal surface 14 of the frame 20 can be coated with a first layer 30 comprising the therapeutic agent, and a second layer 40 positioned over at least the first layer 30. The second layer 40 comprises a bioabsorbable elastomer. Preferably, the first layer 30 consists essentially of a hydrophobic therapeutic agent releasibly adhered to at least a portion of the abluminal surface 14 of an implantable medical device frame 20, and positioned between the abluminal surface 14 of the implantable medical device 20 and a second layer 40 consisting essentially of a bioabsorbable elastomer material. The first layer 30 preferably contains a therapeutically effective amount of the therapeutic agent. More preferably, the first layer 30 is substantially free of a polymer, such as the biodegradable elastomer present in the second layer 40. The second layer 40 can be positioned over at least the first layer 30 and is optionally positioned over all or part of the abluminal surface of the medical device. Also preferably, the second layer 40 can be substantially free of the therapeutic agent. For example, the second layer 40 may consist essentially of a biodegradable elastomer containing less than about 0.1 mg of the therapeutic agent per mm² surface area of the second layer 40.

The first layer 30 and the second layer 40 can have any suitable thickness. FIG. 1B shows a cross section of a coated portion of the frame 20 along the line A-A′ in FIG. 1A, including the luminal surface 14 and the abluminal surface 16. In the embodiment illustrated in FIG. 1B, the first layer 30 can consist essentially of a hydrophobic therapeutic agent adhered directly to the abluminal surface of the frame 20, and the second layer 40 positioned over both the first layer 30 and the luminal surface 16 of the frame 20. Alternatively, as shown in the cross sectional view of the frame 20 along the line A-A′ of FIG. 1C, the second layer 40 can be deposited over only the luminal surface 14 of the frame 20, without being deposited over the abluminal surface 16 of the frame 20. Preferably, the second layer 40 encloses the first layer 30, such that the exterior surface of the coating does not include the uncovered therapeutic agent prior to elution of the therapeutic agent.

The coating can be applied to any suitable surface of a medical device, including on substantially flat or roughened metal surfaces, impregnation within tissue grafts or polymer gels, within grooves, holes or wells formed in portions of a device. The medical device is preferably configured as a vascular stent or stent graft, although the coatings can be applied to any suitable implantable medical device. For example, implantable portions of catheters, billiary or urological stents or shunts, stent grafts, tissue grafts, orthopedic implants, pacemakers, implantable valves and other implantable devices can be coated with the coatings disclosed herein, so as to release a therapeutic agent upon implantation.

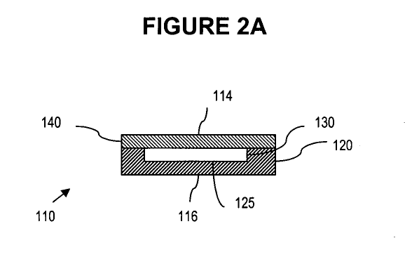

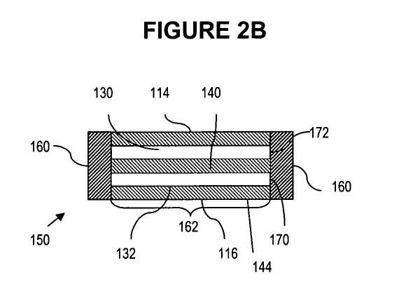

In other embodiments, the invention may include a layer(s) in which the therapeutic agent is contained within the medical device itself. The medical device may have holes, wells, slots, grooves, or the like for containing the therapeutic agent and/or polymer (see, e.g., co-pending U.S. application Ser. No. 10/870,079, incorporated herein by reference). FIG. 2A shows a cross section of a coated portion of a medical device 110, such as a modified version of the medical device of FIG. 1A along the line A-A′. The medical device includes a frame 120 that has a well 125 that contains the first layer 130 comprising a therapeutic agent. The first layer 130 is similar to the first layer 30 described above, except that it is positioned within the well 125 instead of above the surface of the medical device 110. The first layer 130 is enclosed by the walls of the well 125 and the second layer 140. The well 125 can have any suitable dimensions, and can be formed in the medical device by any suitable method, including the mechanical or chemical removal of portions of the medical device frame. A second layer 140 comprising a bioabsorbable elastomer is positioned over the first layer 130 and on the abluminal surface 114 of the medical device 110. The second layer 140 is similar to the second layer 40 described above. The luminal surface 116 of the medical device 110 can be uncoated. Alternatively, the therapeutic agent and/or bioabsorbable elastomer may be incorporated into a biodegradable medical device frame 120 that releases the therapeutic agent as the device degrades, or the therapeutic agent and/or bioabsorbable elastomer may be incorporated into or placed on the medical device frame 120 in any other known manner.

Optionally, the medical device coating can further include more than two layers. FIG. 2B shows a cross section of a coated portion of a medical device 150, such as a modified version of the medical device of FIG. 1A along the line A-A′. The medical device includes a frame 160 that has a hole 162 extending between the luminal surface 116 and the abluminal surface 114 that contains a plurality of layers 170 within the hole 162. The layers 170 include a first therapeutic layer 130 and a second therapeutic layer 132 that comprise the same of different therapeutic agent(s). For example, a first coating layer 140 can be positioned between the first therapeutic layer 130 and the second therapeutic layer 132, a second coating layer 142 can be positioned on the abluminal side of the first therapeutic layer 130 and a third coating layer 144 can be positioned on the luminal side of the second therapeutic layer 132. The first coating layer 140, the second coating layer 142 and the third coating layer 144 can have compositions and thicknesses that are the same or different. Preferably, the first coating layer 140 and the second coating layer 142 include a bioabsorbable elastomer. The third coating layer 144 can include a material that functions to direct the elution of the therapeutic agent toward the abluminal surface 114 of the medical device, or to slow the elution rate of the therapeutic agent elution on the luminal side. The second coating layer 142 can be formed from a bioabsorbable material that is more porous than or degrades more rapidly than the first coating layer 140 upon implantation. Accordingly, the first therapeutic layer 130 can elute from the abluminal surface 114 before or more rapidly than the second therapeutic layer 132. The rapid elution of the first therapeutic layer 130 can provide an initial “burst” of the therapeutic agent to a portion of the body vessel contacting the abluminal surface 114, followed by a more gradual and sustained elution from the second therapeutic layer 132 to the abluminal surface 114.

The plurality of layers 170 in the coating can include any suitable numbers of layers comprising the therapeutic agent and layers comprising other coating materials such as bioabsorbable elastomers, including 2, 3, 4, 5, 6, 7, 8, 9, or 10-layer coatings. Preferably, layers comprising a bioabsorbable elastomer are positioned between layers comprising one or more therapeutic agent(s). Different therapeutic agents can be placed in different layers or within the same layer. Alternatively, a layer such as the third coating layer 144 can be formed from a bioabsorbable material to permit elution of the therapeutic agent toward the luminal surface 116. In yet another alternative coating configuration, the first coating layer 140 can be formed from a biostable material and the second coating layer 142 and the third coating layer 144 can be formed from a bioabsorbable elastomer permitting elution of the first therapeutic layer 130 from the abluminal surface 114 and the second therapeutic layer 132 from the luminal surface 116.

In other embodiments, additional layers other than layers containing the therapeutic agent or the bioabsorbable elastomer can be placed between the first layer comprising the therapeutic agent and the surface of the medical device, between the first layer and the second layer or over the second layer. The optional additional layers can, for example, promote adhesion of the therapeutic agent to the medical device or to desirably affect the release of the therapeutic agent. For example, an adhesion promoting layer can be deposited between the frame 160 in FIG. 2B and the plurality of layers 170. The adhesion promoting layer can be formed from any suitable material that promotes the adhesion or retention of one or more of the coating layers, such as silane, pyrolytic carbon, parylene and the like.

In some embodiments, materials that promote the adhesion of an outer coating layer, such as coating layer 142 in FIG. 2A, to the wall of a body vessel. Alternatively, materials that promote adhesion to a portion of the body upon implantation therein can be incorporated into the coating layer 142. Chemical or biological modifications of the device surface or coating layers can also enhance adhesion between an implantable medical device and the surrounding host tissue. For example, devices have been coated with a substance to enhance the healing process and/or adhesion of the device to the host tissue. In one approach, implantable medical devices can permit infiltration by specific desirable tissue cells. One type of tissue infiltration involves the process known as “endothelialization”, i.e., migration of endothelial cells from adjacent tissue onto or into the device surface. Methods for promoting endothelialization can include applying a porous coating to the device which allows tissue growth into the interstices of the implant surface (see, e.g., WO 96/37165A1). Also, an electrically charged or ionic material (e.g., fluoropolymer) can be applied to a portion of the tissue-contacting surface of the device or device coating (see, e.g., WO 95/19796A1; J. E. Davies, in Surface Characterization of Biomaterials, B. D. Ratner, ed., pp. 219-234 (1988); and U.S. Pat. No. 5,876,743). Biocompatible organic polymers (e.g., polymers substituted with carbon, sulfur or phosphorous oxyacid groups) can be added to a coating layer or portions of the medical device frame to promote osteogenesis at the host-implant interface (see, e.g., U.S. Pat. No. 4,795,475), or coatings made from biological materials (e.g., collagen) can be used to enhance tissue repair, growth and adaptation at the implant-tissue interface (e.g., U.S. Pat. No. 5,002,583).

Therapeutic Agent Elution Profiles

Medical device coatings may be characterized by measuring the elution profile of the coating in a particular elution medium. An elution profile is a graph showing the rate at which the therapeutic agent is released from a coated medical device into an elution medium as a function of time the coating is in contact with an elution medium. Elution profiles may be used to identify particularly preferred coating configurations that provide a release of a therapeutic agent at a desired rate and/or for a desired period of time. Sustained release coatings characterized by a release of about 70-90% of the therapeutic agent from the coating over a period of about 15-20 days in porcine serum are particularly preferred for some applications. Desirably, the coatings are also configured to release a therapeutically effective dose of the therapeutic agent over a treatment period. The treatment period for restenosis may vary, but can be about 15 days for delivery of about 10-15 mg of a taxane therapeutic agent to a portion of an arterial wall.

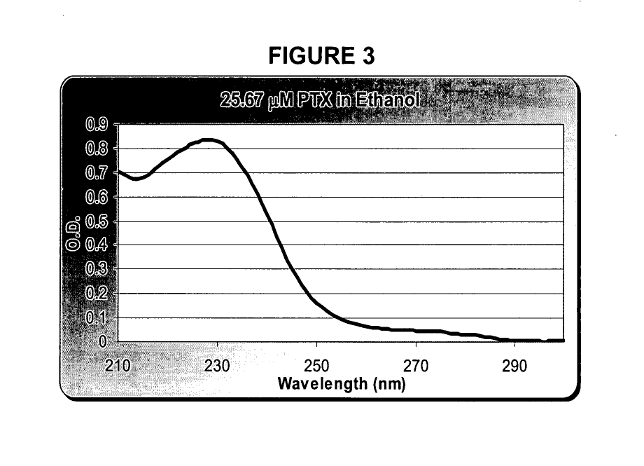

The amount of therapeutic agent released from coating into the elution medium, and the rate of release of the therapeutic agent from a coating, can be measured by any suitable method that allows for measurement of the release of the therapeutic agent with a desired level of accuracy and precision. The therapeutic agent in the coating can be determined by dissolving the coating in a suitable elution medium and subsequently detecting the amount of therapeutic agent in the elution medium. The therapeutic agent dissolved in the elution medium can be detected using any suitable technique. A suitable method, such as a spectrographic technique, permits measurement of a property of the test solution that can be correlated to the presence or concentration of the therapeutic agent analyte with a desired level of accuracy and precision. Various spectrographic measurements of the elution medium can be correlated with the amount of therapeutic agent removed from the medical device coating. Suitable spectrographic techniques for detecting the therapeutic agent in the elution medium include: UV absorption spectrum of a elution medium after contacting the medical device, use of an HPLC spectrophotometer with to a UV-VIS detector, or Liquid Chromotagrphy paired with a Mass Spectrophotometer Detector. For example, taxane therapeutic agents, such as paclitaxel, can be detected in a porcine serum elution medium using a UV-Visible Spectrophotometer. The detection of the therapeutic agent can be correlated to the amount of therapeutic agent that was present on the medical device surface prior to contacting the medical device with the solvent. When absorption spectroscopy is used to detect the presence of a therapeutic agent, such as in a test solution or solvent solution, the Beer-Lambert Correlation can be used to determine the concentration of a therapeutic agent in the solution. This correlation involves the linear relationship between optical density (absorbance) and concentration of an absorbing species. Using a set of standard samples with known concentrations, the correlation can be used to measure the optical density (O.D.) of the sample. A plot of concentration versus optical density (calibration plot) can then be used to determine the concentration of an unknown solution from its optical density. FIG. 3 shows a UV-Visible Spectra for 25.67 μM paclitaxel in an ethanol elution medium. The presence of paclitaxel and certain taxanes can be detected in the porcine serum based on the absorption at about 230 nm. Such data may be obtained from an apparatus such as the Agilent 8453 Phtodiode Array UV-Vis Spectrophotometer. A calibration plot can be made by measuring the optical density of known concentrations of a therapeutic agent. Then, a coated medical device comprising an unknown amount of therapeutic agent can be placed in contact with the elution medium to dissolve the therapeutic agent at a desired rate, and subsequent detection of the optical density of the therapeutic agent in the elution medium can be correlated to the amount of therapeutic agent coated on or dissolved from the medical device coating.

The elution profile of a coated medical device can vary depending on the elution medium and conditions in which the therapeutic agent is released. A suitable elution medium solubilizes a therapeutic agent while allowing for subsequent measurement of the solubilized therapeutic agent in a manner that can be correlated to the amount of therapeutic agent in the coating. Preferably, substantially all of the therapeutic agent is removed from the medical device after contact with the elution medium for a desired period of time. The desired time period for elution should be long enough to permit adequate resolution in measurement of the release rate into the elution medium, but short enough not to require an undesirably long period of time to measure the total amount of the therapeutic agent in the coating.

The elution profile of a medical device coating can be measured in vitro by performing an elution assay. An elution assay measures the drug elution profile of a coated medical device. Different elution media can be used that provide desired rates of drug elution. For example, an elution medium such as SDS can be selected to quickly dissolve a hydrophobic therapeutic agent in the coating, for example to measure the total amount of therapeutic agent. Alternatively, an elution medium such as porcine serum can be selected to gradually dissolve the hydrophobic therapeutic agent over a much longer period time, for example to measure the rate of release of the therapeutic agent. For purposes of this application, unless otherwise specified, the elution profile of a therapeutic agent was obtained in vitro by contacting the medical device with a modified porcine elution medium prepared by adding 0.104 mL of a 6.0 g/L Heparin solution to porcine serum and adjusting the pH to 5.6±0.3 using a 20% v/v aqueous solution of acetic acid. This modified procine serum elution medium provides for the gradual release of the therapeutic agent at a rate that is similar to blood. Alternatively, other elution media can be used to more rapidly dissolve the therapeutic agent.

Therapeutic Agents

An implantable medical device may comprise a therapeutically effective amount of one or more therapeutic agents in one or more layers. The therapeutic agent can be selected to treat a desired clinical indication. The therapeutically effective amount of therapeutic agent can depend upon the condition and severity of the condition to be treated; the type and activity of the specific therapeutic agent employed; the method by which the medical device is administered to the patient; the age, body weight, general health, gender and diet of the patient; the time of administration, route of administration, and rate of excretion of the specific compound employed; the duration of the treatment; drugs used in combination or coincidental with the specific compound employed; and like factors well known in the medical arts. For instance, a coating layer comprising a therapeutic agent may include 0.01, 0.05, 0.10, 0.20, 0.25, 0.30, 0.40, 0.50, 0.60, 0.70, 0.75, 0.80, 0.90 1.00, 1.25, 1.50, 1.75, 2.00, 2.25, 2.50, 2.75, 3.00, 3.25, 3.50, 3.75 and 4.00 μg/mm² of the taxane therapeutic agent, including intervals of about 0.01 and 0.001 therebetween. The coating preferably includes at least one layer comprising between about 0.01-4.00 μg/mm², 0.03-3.00 μg/mm², 0.05-2.00 μg/mm², and more preferably about 0.01-1.00 μg/mm². For taxane therapeutic agents in particular, the coating preferably includes a first layer comprising or consisting essentially of about 0.05-1.00 μg/mm², more preferably about 0.05-0.90 μg/mm², 0.06-μg/mm², 0.30 μg/mm², or 90 μg/mm² of the taxane therapeutic agent on the first layer. Preferably, a first layer comprising the therapeutic agent(s) is positioned over a portion of a surface area of a medical device configured to contact the wall of a body vessel. Desirably, a total of about 0.01-4.00 μg of a taxane therapeutic agent per mm² is positioned on the abluminal surface area of the vascular stent.

The layer(s) comprising the therapeutic agent preferably do not contain material, such as a polymer, that may alter the solubility properties of the therapeutic agent. Most preferably, the coating includes one or more layer(s) consisting of the therapeutic agent, or consisting essentially of the therapeutic agent. Accordingly, layer(s) comprising the therapeutic agent typically contain less than about 10 μg, or more preferably less than about 1, 0.50, 0.25, or 0.10 μg, of the bioabsorbable elastomer per mm² of the total surface area of the layer. The thickness of the layer may be selected to provide a desired rate of release. Each layer comprising a therapeutic agent preferably has a thickness of about 0.2 μm to about 10 μm, and more preferably about 0.2 μm to 5 μm.

Preferably, the therapeutic agent is sparingly soluble or insoluble in water. For example, the therapeutic agent can be a hydrophobic compound, preferably having a solubility in water that is about 0.25 mg/mL, and more preferably less than about 0.20, 0.10, 0.05, 0.02 or 0.01 mg/mL water. The therapeutic agent may be provided in any suitable form, including a pharmaceutically acceptable salt, as a prodrug, or as a derivative or analog of a compound named herein, or equivalents thereto.

Therapeutic agents that may be used in the present invention include, but are not limited to, pharmaceutically acceptable compositions containing any of the therapeutic agents or classes of therapeutic agents listed herein, as well as any salts and/or pharmaceutically acceptable formulations thereof. Table 1 below provides a non-exclusive list of classes of therapeutic agents and some corresponding exemplary active ingredients.

TABLE 1

| Therapeutic Agent Class | Exemplary Active Ingredients |
|---|---|
| Adrenergic agonist | Adrafinil |
| | Isometheptene |
| | Ephedrine (all forms) |
| Adrenergic antagonist | Monatepil maleate |
| | Naftopidil |
| | Carvedilol |
| | Moxisylyte HCl |
| Adrenergic - Vasoconstrictor/Nasal | Oxymetazoline HCl |
| decongestant | Norfenefrine HCl |
| | Bretylium Tosylate |
| Adrenocorticotropic hormone | Corticotropin |
| Analgesic | Bezitramide |
| | Acetylsalicysalicylic acid |
| | Propanidid |
| | Lidocaine |
| | Pseudophedrine hydrochloride |
| | Acetominophen |
| | Chlorpheniramine Maleate |
| Anesthetics | Dyclonine HCl |
| | Hydroxydione Sodium |
| | Acetamidoeugenol |
| Anthelmintics | Niclosamide |
| | Thymyl N-Isoamylcarbamate |
| | Oxamniquine |
| | Nitroxynil N-ethylglucamine |
| | Anthiolimine |
| | 8-Hydroxyquinoline Sulfate |
| Anti-inflammatory | Bendazac |
| | Bufexamac |
| | Desoximetasone |
| | Amiprilose HCl |
| | Balsalazide Disodium Salt |
| | Benzydamine HCl |
| | Corticosteroids (Methylprednisolone, |
| | Dexamethasone) |
| | Tranilast (N-(3,4-dimethoxycinnamoyl) |
| | anthranilic acid) |
| Antiallergic | Fluticasone propionate |
| | Pemirolast Postassium salt |
| | Cromolyn Disodium salt |
| | Nedocromil Disodium salt |
| Antiamebic | Cephaeline |
| | Phanquinone |
| | Thiocarbarsone |
| Antianemic | Folarin |
| | Calcium folinate |
| Antianginal | Verapamil |
| | Molsidomine |
| | Isosorbide Dinitrate |
| | Acebutolol HCl |
| | Bufetolol HCl |
| | Timolol Hydrogen maleate salt |
| Antiarryhythmics | Quinidine |
| | Lidocaine |
| | Capobenic Acid |
| | Encainide HCl |
| | Bretylium Tosylate |
| | Butobendine Dichloride |
| Antiarthritics | Azathioprine |
| | Calcium 3-aurothio-2-propanol-1-sulfate |
| | Glucosamine Beta Form |
| | Actarit |
| Antiasthmatics/Leukotriene antagonist | Cromalyn Disodium |
| | Halamid |
| | Montelukast Monosodium salt |
| Antibacterial | Cefoxitin Sodium salt |
| | Lincolcina |
| | Colisitin sulfate |
| Antibiotics | Gentamicin |
| | Erythromycin |
| | Azithromycin |
| Anticoagulants | Heprin sodum salt |
| | Heprinar |
| | Dextran Sulfate Sodium |
| Anticonvulsants | Paramethadione |
| | Phenobarbital sodium salt |
| | Levetiracetam |
| Antidepressants | Fluoxetine HCl |
| | Paroxetine |
| | Nortiptyline HCl |
| Antidiabetic | Acarbose |
| | Novorapid |
| | Diabex |
| Antiemetics | Chlorpromazine HCl |
| | Cyclizine HCl |
| | Dimenhydrinate |
| Antiglaucoma agents | Dorzolamide HCl |
| | Epinepherine (all forms) |
| | Dipivefrin HCl |
| Antihistamines | Histapyrrodine HCl |
| Antihyperlipoproteinemic | Lovastatin |
| | Pantethine |
| Antihypertensives | Atenolol |
| | Guanabenz Monoacetate |
| | Hydroflumethiazide |
| Antihyperthyroid | Propylthiouracil |
| | Iodine |
| Antihypotensive | Cortensor |
| | Pholedrine Sulfate |
| | Norepinephrine HCl |
| Antimalarials | Cinchonidine |
| | Cinchonine |
| | Pyrimethamine |
| | Amodiaquin Dihydrochloride dihydrate |
| | Bebeerine HCl |
| | Chloroquine Diphosphate |
| Antimigraine agents | Dihydroergotamine |
| | Ergotamine |
| | Eletriptan Hydrobromide |
| | Valproic Acid Sodium salt |
| | Dihydroergotamine mesylate |
| Antineoplastic | 9-Aminocamptothecin |
| | Carboquone |
| | Benzodepa |
| | Bleomycins |
| | Capecitabine |
| | Doxorubicin HCl |
| Antiparkinsons agents | Methixene |
| | Terguride |
| | Amantadine HCl |
| | Ethylbenzhydramine HCl |
| | Scopolamine N-Oxide Hydrobromide |
| Antiperistaltic; antidiarrheal | Bismuth Subcarbonate |
| | Bismuth Subsalicylate |
| | Mebiquine |
| | Diphenoxylate HCl |
| Antiprotozoal | Fumagillin |
| | Melarsoprol |
| | Nitazoxanide |
| | Aeropent |
| | Pentamideine Isethionate |
| | Oxophenarsine Hydrochloride |
| Antipsycotics | Chlorprothixene |
| | Cyamemazine |
| | Thioridazine |
| | Haloperidol HCl |
| | Triflupromazine HCl |
| | Trifluperidol HCl |
| Antipyretics | Dipyrocetyl |
| | Naproxen |
| | Tetrandrine |
| | Imidazole Salicylate |
| | Lysine Acetylsalicylate |
| | Magnesium Acetylsalicylate |
| Antirheumatic | Auranofin |
| | Azathioprine |
| | Myoral |
| | Penicillamine HCl |
| | Chloroquine Diphosphate |
| | Hydroxychloroquine Sulfate |
| Antispasmodic | Ethaverine |
| | Octaverine |
| | Rociverine |
| | Ethaverine HCl |
| | Fenpiverinium Bromide |
| | Leiopyrrole HCl |
| Antithrombotic | Plafibride |
| | Triflusal |
| | Sulfinpyrazone |
| | Ticlopidine HCl |
| Antitussives | Anethole |
| | Hydrocodone |
| | Oxeladin |
| | Amicibone HCl |
| | Butethamate Citrate |
| | Carbetapentane Citrate |
| Antiulcer agents | Polaprezinc |
| | Lafutidine |
| | Plaunotol |
| | Ranitidine HCl |
| | Pirenzepine 2 HCl |
| | Misoprostol |
| Antiviral agents | Nelfinavir |
| | Atazanavir |
| | Amantadine |
| | Acyclovir |
| | Rimantadine HCl |
| | Epivar |
| | Crixivan |
| Anxiolytics | Alprazolam |
| | Cloxazolam |
| | Oxazolam |
| | Flesinoxan HCl |
| | Chlordiazepoxide HCl |
| | Clorazepic Acid Dipotassium salt |
| Broncodialtor | Epinephrine |
| | Theobromine |
| | Dypylline |
| | Eprozinol 2HCl |
| | Etafedrine |
| Cardiotonics | Cymarin |
| | Oleandrin |
| | Docarpamine |
| | Digitalin |
| | Dopamine HCl |
| | Heptaminol HCl |
| Cholinergic | Eseridine |
| | Physostigmine |
| | Methacholine Chloride |
| | Edrophonium chloride |
| | Juvastigmin |
| Cholinergic antagonist | Pehencarbamide HCl |
| | Glycopyrrolate |
| | Hyoscyamine Sulfate dihydrate |
| Cognition enhancers/Nootropic | Idebenone |
| | Tacrine HCl |
| | Aceglutamide Aluminum Complex |
| | Acetylcarnitine L HCl |
| Decongestants | Propylhexedrine dl-Form |
| | Pseudoephedrine |
| | Tuaminoheptane |
| | Cyclopentamine HCL |
| | Fenoxazoline HCl |
| | Naphazoline HCl |
| Diagnostic aid | Disofenin |
| | Ethiodized Oil |
| | Fluorescein |
| | Diatrizoate sodium |
| | Meglumine Diatrizoate |
| Diuretics | Bendroflumethiazide |
| | Fenquizone |
| | Mercurous Chloride |
| | Amiloride HCl 2 H₂O |
| | Manicol |
| | Urea |
| Enzyme inhibitor (proteinase) | Gabexate Methanesulfonate |
| Fungicides | Candicidin |
| | Filipin |
| | Lucensomycin |
| | Amphotericin B |
| | Caspofungin Acetate |
| | Viridin |
| Gonad stimulating principle | Clomiphene Citrate |
| | Chorionic gonadotropin |
| | Humegon |
| | Luteinizing hormone (LH) |
| Hemorheologic agent | Poloxamer 331 |
| | Azupentat |
| Hemostatic | Hydrastine |
| | Alginic Acid |
| | Batroxobin |
| | 6-Aminohexanoic acid |
| | Factor IX |
| | Carbazochrome Salicylate |
| Hypolimpemic agents | Clofibric Acid Magnesium salt |
| | Dextran Sulfate Sodium |
| | Meglutol |
| Immunosuppresants | Azathioprine |
| | 6-Mercaptopurine |
| | Prograf |
| | Brequinar Sodium salt |
| | Gusperimus Trihydrochloride |
| | Mizoribine |
| | FK106 (Tacrolimus) |
| | Mycophenolic acid (MPA) |
| | mTOR Inhibitors, including Rapamycin and |
| | analogs thereof, such as: |
| | Sirolimus, |
| | Everolimus ([40-O-(2-hydroxyethyl)- |
| | rapamycin]), and |
| | ABT-578 (methyl rapamycin) |
| Mydriatic; antispasmodic | Epinephrine |
| | Yohimbine |
| | Aminopentamide dl-Form |
| | Atropine Methylnitrate |
| | Atropine Sulfatemonohydrate |
| | Hydroxyamphetamine (l, HCl, HBr) |
| Neuromuscular blocking agent/ | Phenprobamate |
| Muscle relaxants (skeletal) | Chlorzoxazone |
| | Mephenoxalone |
| | Mioblock |
| | Doxacurium Chloride |
| | Pancuronium bromide |
| Oxotocic | Ergonovine Tartrate hydrate |
| | Methylergonovine |
| | Prostaglandin F_{2α} |
| | Intertocine-S |
| | Ergonovine Maleate |
| | Prostoglandin F_{2$$} Tromethamine salt |
| Radioprotective agent | Amifostine 3H₂O |
| Sedative/Hypnotic | Haloxazolam |
| | Butalbital |
| | Butethal |
| | Pentaerythritol Chloral |
| | Diethylbromoacetamide |
| | Barbital Sodium salt |
| Serenic | Eltoprazine |
| Tocolytic agents | Albuterol Sulfate |
| | Terbutaline sulfate |
| Treatment of cystic fibrosis | Uridine 5′-Triphosphate Trisodium dihydrate |
| | salt |
| Vasoconstrictor | Nordefrin (—) Form |
| | Propylhexedrine dl-form |
| | Nordefrin HCl |
| Vasodilators | Nylidrin HCl |
| | Papaverine |
| | Erythrityl Tetranitrate |
| | Pentoxifylline |
| | Diazenium diolates |
| | Citicoline |
| | Hexestrol Bis($$-diethylaminoethyl ether) 2HCl |
| Vitamins | α-Carotene |
| | β-Carotene |
| | Vitamin D₃ |
| | Pantothenic Acid sodium salt |

Within some preferred embodiments of the invention, the therapeutic agent is a taxane cell cycle inhibitor, such as paclitaxel, a paclitaxel analogue or paclitaxel derivative compound. Paclitaxel is a bioactive compound which disrupts mitosis (M-phase) by binding to tubulin to form abnormal mitotic spindles or an analogue or derivative thereof. Briefly, paclitaxel is a highly derivatized diterpenoid (Wani et al., J. Am. Chem. Soc. 93: 2325, 1971) which has been obtained from the harvested and dried bark of Taxus brevifolia (Pacific Yew) and Taxomyces Andreanae and Endophytic Fungus of the Pacific Yew (Stierle et al., Science 60: 214-216, 1993).

The term “Paclitaxel” refers herein to a compound of the chemical structure shown as structure (1) below, consisting of a core structure with four fused rings (“core taxane structure,” shaded in structure (1)), with several substituents.

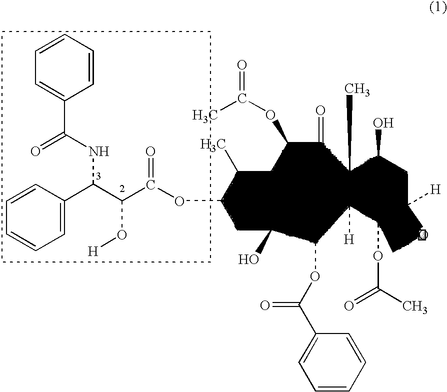

In another embodiment, the therapeutic agent can be a taxane analog or derivative characterized by variation of the paclitaxel structure (1). Taxanes in general, and paclitaxel is particular, is considered to function as a cell cycle inhibitor by acting as an anti-microtubule agent, and more specifically as a stabilizer. Preferred taxane analogs and derivatives core vary the substituents attached to the core taxane structure. In one embodiment, the therapeutic agent is a taxane analog or derivative including the core taxane structure (1) and the methyl 3-(benzamido)-2-hydroxy-3-phenylpropanoate moiety (shown in structure (2) below) at the 13-carbon position (“C13”) of the core taxane structure (outlined with a dashed line in structure (1)). 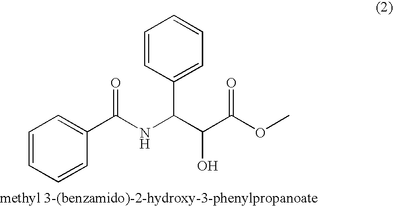
It is believed that structure (2) at the 13-carbon position of the core taxane structure plays a role in the biological activity of the molecule as a cell cycle inhibitor. Examples of therapeutic agents having structure (2) include paclitaxel (Merck Index entry 7117), docetaxol (TAXOTERE, Merck Index entry 3458), and 3′-desphenyl-3′-(4-ntirophenyl)-N-dibenzoyl-N-(t-butoxycarbonyl)-10-deacetyltaxol.

A therapeutic agent composition comprising a taxane compound can include formulations, prodrugs, analogues and derivatives of paclitaxel such as, for example, TAXOL (Bristol Myers Squibb, New York, N.Y., TAXOTERE (Aventis Pharmaceuticals, France), docetaxel, 10-desacetyl analogues of paclitaxel and 3′N-desbenzoyl-3′N-t-butoxy carbonyl analogues of paclitaxel. Paclitaxel has a molecular weight of about 853 amu, and may be readily prepared utilizing techniques known to those skilled in the art (see, e.g., Schiff et al., Nature 277: 665-667, 1979; Long and Fairchild, Cancer Research 54: 4355-4361, 1994; Ringel and Horwitz, J. Nat'l Cancer Inst. 83 (4): 288-291, 1991; Pazdur et al., Cancer Treat. Rev. 19 (4): 351-386, 1993; WO 94/07882; WO 94/07881; WO 94/07880; WO 94/07876; WO 93/23555; WO 93/10076; WO94/00156; WO 93/24476; EP 590267; WO 94/20089; U.S. Pat. Nos. 5,294,637; 5,283,253; 5, 279,949; 5,274,137; 5,202,448; 5,200,534; 5,229,529; 5,254,580; 5,412,092; 5,395,850; 5,380,751; 5,350,866; 4,857,653; 5,272,171; 5,411,984; 5,248, 796; 5,248,796; 5,422,364; 5,300,638; 5,294,637; 5,362,831; 5,440,056; 4, 814,470; 5,278,324; 5,352,805; 5,411,984; 5,059,699; 4,942,184; Tetrahedron Letters 35 (52): 9709-9712,1994; J. Med. Chem. 35: 4230-4237, 1992; J. Med. Chem. 34: 992-998, 1991; J. Natural Prod. 57 (10): 1404-1410, 1994; J. Natural Prod. 57 (11): 1580-1583,1994; J. Am. Chem. Soc. 110: 6558-6560, 1988), or obtained from a variety of commercial sources, including for example, Sigma Chemical Co., St. Louis, Mo. (T7402—from Taxus brevifolia).

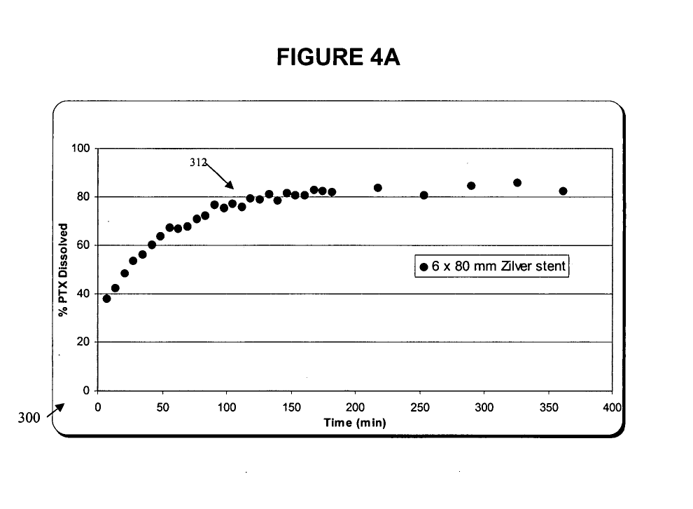

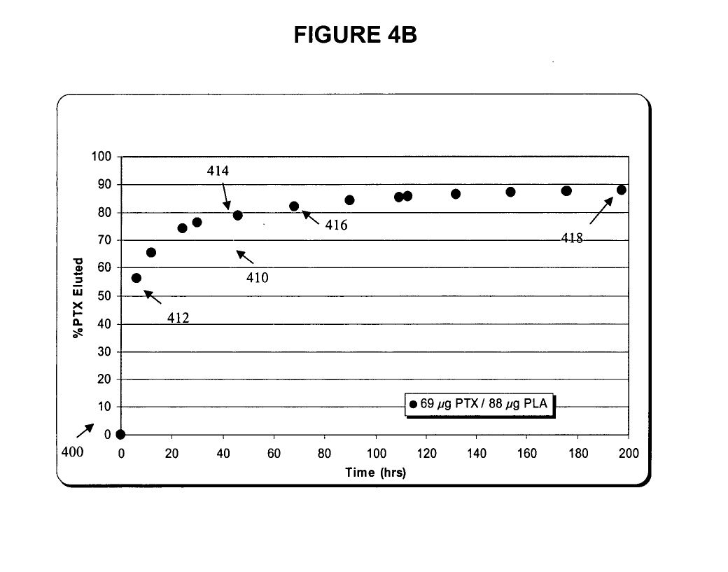

FIG. 4A shows the elution profile of a first vascular stent having a single layer coating consisting of 961 μg of paclitaxel on a 6×80 mm the abluminal side of a self-expanding cylindrical nickel-titanium alloy (NITINOL) Zilver® stent (shown in FIG. 1A). The abluminal surface area of the coated stent was about 288 mm², and the paclitaxel was coated as a single layer at a dose of about 3 mg/mm² of the abluminal surface area. No biodegradable elastomer coating layer was present on the stent. The paclitaxel was coated onto the abluminal surface of the stent by spraying a 4.68 mM paclitaxel ethanol solution of paclitaxel onto the stent and allowing the ethanol to evaporate. Elution profile 300 shows the percent of the paclitaxel eluted from a paclitaxel-coated as a function of time in a porcine serum elution medium. The elution profile 300 was obtained by contacting the paclitaxel coated stent with a modified porcine serum elution medium at a constant flow rate of 16 mL/min for about 6 hours. The percentage of paclitaxel dissolved was measured as a function of time by monitoring the optical density of the elution medium after contacting the coated stent, as described above. The modified porcine serum elution medium was prepared by adding 0.104 mL of a 6.0 g/L Heparin solution to porcine serum at 37° C. and adjusting the pH to 5.6+/−0.3 using a 20% v/v aqueous solution of acetic acid. The elution rate profile 300 of the paclitaxel includes a first rate of drug release over an initial period of about 2 hours (120 minutes) after stent contact with the porcine serum, and a second, slower rate of drug elution over the next several hours (120-350 minutes). After about 90 minutes in contact with the porcine serum (point 312), about 75% of the paclitaxel had eluted from the coating at the first rate of drug release.

However, for some applications, local administration of therapeutic agents from a medical device coating may be more effective when carried out over a longer period of time, such as a time period at least matching the normal reaction time of the body to an angioplasty procedure, for example. For example, local administration of a therapeutic agent over a period of days or even months may be most effective in treating or inhibiting conditions such as restenosis. Different coating configurations may be selected to provide different rates of release of a therapeutic agent from a medical device.

Coating Configurations and Elution Rates

The elution profile of a medical device can be altered by varying the composition and/or thickness of the coating layers and the ratio of the therapeutic agent to the bioabsorbable elastomer. For example, the elution rate can be decreased by (1) increasing the weight ratio of the the bioasborbable elastomer to the therapeutic agent, (2) increasing the relative thickness of a bioabsorbable elastomer coating layer to an adjacent underlying therapeutic agent coating layer, and (3) decreasing the total amount of therapeutic agent in a coating layer.

The coating layers comprising the bioabsorbable elastomer are preferably thick enough to provide a desired rate of release of the therapeutic agent, but thin enough to provide a desired level of durability of the overall coating. Increasing the thickness of the bioabsorbable elastomer or increasing the weight ratio of the bioabsorbable elastomer to the therapeutic agent can decrease the rate of elution measured in the elution profile. Desirably, the thickness of each layer comprising the therapeutic agent or the biodegradable elastomer is selected to provide a desired rate of release of the therapeutic agent for an intended use. However, if the thickness of a layer is too large, however, the durability of the coating may be decreased.

Preferably, a coating layer comprising a biodegradable elastomer has a greater amount of biodegradable elastomer by weight than the weight of therapeutic agent in an adjacent coating layer. For example, the total weight ratio of therapeutic agent to bioabsorbable elastomer in an adjacent layer is preferably about 1:1 to about 1:100, including ratios of 1:5, 1:10, 1:25, 1:50, and 1:75 (including all ratios therebetween), measured as a total weight ratio of an entire coating having one or more layers. Preferably, the weight ratio of the amount of therapeutic agent to bioabsorbable elastomer in an adjacent layer is about 1:1 to about 1:20.

Preferably, coating layers comprising the bioabsorbable elastomer include a negligible amount of the therapeutic agent, although alternative embodiments can include coating layers with a mixture of the therapeutic agent and the biobabsorbable elastomer. A coating layer comprising the bioabsorbable elastomer preferably contains less than about 10 μg, or more preferably less than about 5, 4, 3, 2, 1, 0.5, 0.25, 0.20, 0.15, 0.10, 0.05 or 0.01 μg, of the therapeutic agent per mm² of the total surface area of the coating layer.

A coating layer comprising a biodegradable elastomer polymer may include an amount of one or more biodegradable elastomer polymer(s) suitable to provide a desired elution rate. For instance, a coating layer may comprise 0.01, 0.05, 0.10, 0.50,1.00, 5.00, 10.00, 15.00, or 20.00 μg /mm² of one or more biodegradable elastomer polymer(s) as a function of the area of the coating layer, including intervals of about 0.01 and 0.001 therebetween. The coating preferably includes at least one layer comprising between about 0.01-20.00 μg/mm², 0.05-5.00 μg/mm², and more preferably about 0.01-3.00 μg/mm² of a biodegradable elastomer polymer. The layer(s) comprising the biodegradable elastomer preferably do not contain a therapeutic agent. Most preferably, the coating includes one or more layer(s) consisting of one or more biodegradable elastomer polymer(s), or consisting essentially of one or more of the biodegradable elastomer polymer(s).

The thickness of the layer may be selected to provide a desired rate of release. Each layer comprising a therapeutic agent preferably has a thickness that is about 2 to 10 times greater than the thickness of an adjacent layer comprising a therapeutic agent. More preferably, the thickness of the bioabsorbable elastomer layer between about 2.0 and 10.0 times greater, preferably about 2.0 to about 5.0 times greater, and most preferably about 2.0 to about 3.0 times greater than the thickness of the therapeutic agent layer(s), including bioabsorbable elastomer layers between about 0.4 μm and about 20 μm. Preferably, the thickness of each therapeutic agent layer can be between about 0.5 μm and about 1.0 μm and the thickness of each bioabsorbable polymer layer is between about 1.0 μm and about 10.0 μm, including bioabsorbable polymer layer thicknesses of about 1.0, 1.5, 2.0, 2.5, 3.0, 3.5, 4.0, 4.5, 5.0, 5.5, 6.0, 6.5, 7.0, 7.5, 8.0, 8.5, 9.0 9.5 and 10.0 μm.

The coating can include any suitable number of layers. The thickness of the entire coating is preferably between about 0.2 μm and about 15 μm. Even more desirably, the thickness of the entire coating is between about 0.6 μm and about 10 μm. For example, for a coating having six layers comprising three layers of therapeutic agent interspersed in an alternating fashion with three layers of polymer, the total thickness of the coating layers would desirably be between about 1.5 μm to about 66.0 μm. Each of the layers can have the same or different thicknesses, with each polymer layer preferably being about 2 to about 10 times thicker than an adjacent layer of therapeutic agent.

The coating can include any suitable number of layers, but preferably includes 2 or more layers, including 2, 3, 4, 5, 6, 7, 8, 9, 10, 11 or 12 layer configurations. The total thickness of the multi-layer coating on any given surface (e.g., luminal or abluminal) of the medical device is preferably between about 0.2 μm and about 75 μm, preferably between about about 0.4 μm and about 50 μm. More preferably, the total thickness of the coating on the abluminal surface is between about 0.5 μm and about 10 μm. The coating may include coating layers consisting essentially of a therapeutic agent or a bioabsorbable elastomer, coating layers containing a mixture of a therapeutic agent and bioabsorbable elastomer, or any combination of these.

The rate of release of the therapeutic agent shown in FIG. 4A may be decreased by applying a second layer comprising a biodegradable elastomer over the therapeutic agent. Preferred coating configurations are characterized by an elution profile that includes a sustained rate of therapeutic agent release over a desired period of time. Accordingly, the coating preferably comprises one or more coating layers comprising a bioabsorbable elastomer polymer covering a therapeutic agent. The coating layer is preferably positioned over a layer of the therapeutic agent. Desirably, the therapeutic agent is completely enclosed by portions of the medical device surface and/or the bioabsorbable elastomer prior to implantation of the coated medical device or contacting the medical device with an elution medium.

Depositing a layer of bioabsorbable elastomer over a layer of therapeutic agent can provide a more sustained release of the therapeutic agent. FIG. 4B shows the elution profile of a second vascular stent. The second coated vascular stent is also a 6×80 mm Zilver® (Cook, Inc., Bloomington, Ind.) having a nickel-titanium alloy frame (NITINOL), but is coated with a two layer coating consisting of a first layer of 69 μg of paclitaxel on the abluminal surface of the vascular stent frame, and a second layer of 88 μg of poly(D,L)-lactic acid (PLA) biodegradable elastomer deposited over the paclitaxel layer. The elution profile 400 shown in FIG. 4B was measured using the same modified porcine serum elution medium, elution conditions and paclitaxel detection methods described above with respect to FIG. 4B, except that the elution of paclitaxel was measured for a much longer period of time (about 200 hours instead of about 6 hours). The elution of the paclitaxel therapeutic agent is indicated as a percentage by weight of total therapeutic agent initially deposited on the stent. The units of therapeutic agent and bioabsorbable elastomer are normalized to micrograms per square millimeter of the abluminal surface area of the stent. Notably, the rate of paclitaxel elution is more gradual than the elution rate measured in FIG. 4A for the similar stent structure coated with over 10-times the amount of paclitaxel, without the the PLA coating. After about 6 hours, a first elution profile point 412 indicates that about 55% of the paclitaxel has eluted from the coated stent, or a rate of about 9.0-10.0% per hour for the first 6 hours. A second elution profile point 414 measured at about 24 hours shown that about 75% of the paclitaxel had eluted, indicating a rate of about 1.0-1.5% per hour between 6-24 hours of elution. A third elution profile point 416 measured at 48 hours shows that about 80% of the paclitaxel has eluted, or a rate of about 0.2% per hour between 24-48 hours of elution. By comparison, about 80% of the paclitaxel was eluted from the first vascular stent elution profile 310 shown in FIG. 4A within about 2 hours. A fourth elution profile point 418 was measured at about 192 hours (about 8 days) showed that nearly 90% of the paclitaxel eluted at this point, or about 10% paclitaxel elution between day 2 through day 8 (about 1.7% per day, or 0.07% per hour). Thus, FIG. 4B shows an elution profile that provides an initial “burst” or rapid release of paclitaxel during the first 6-24 hours, and more gradual sustained release from 24 hours to about 192 hours.

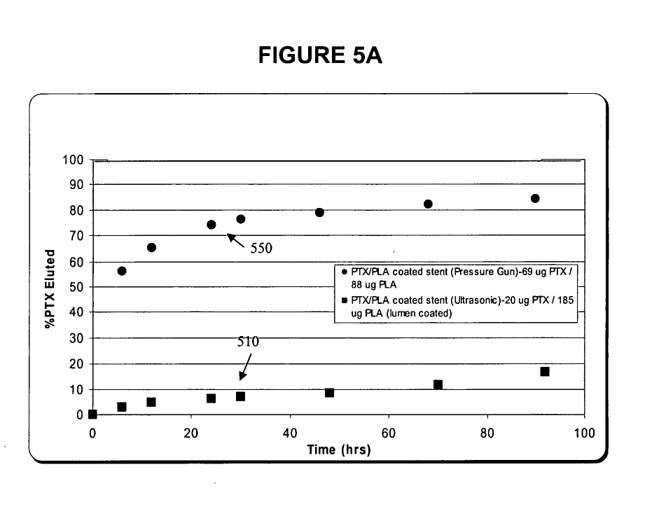

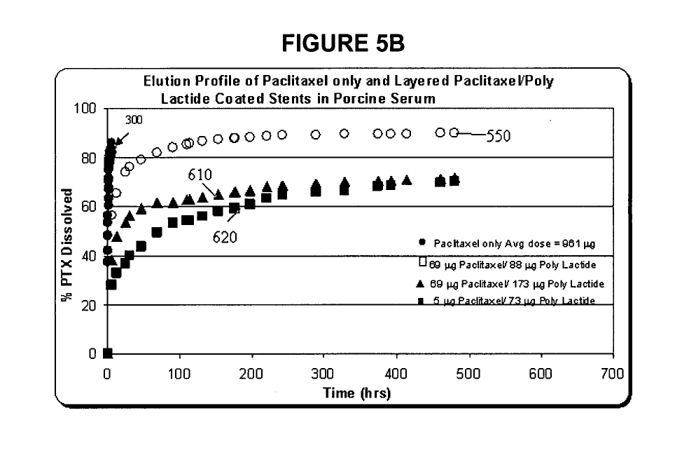

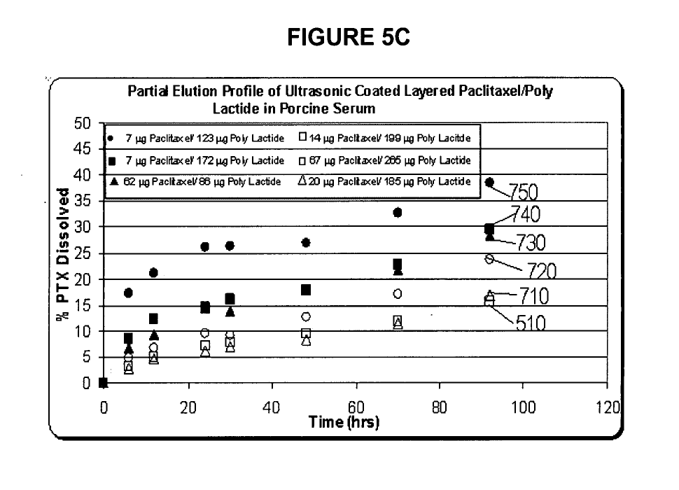

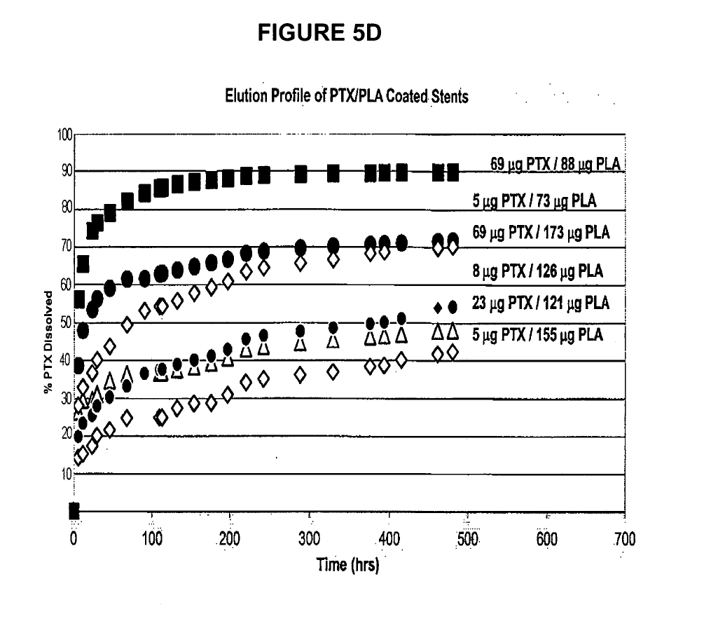

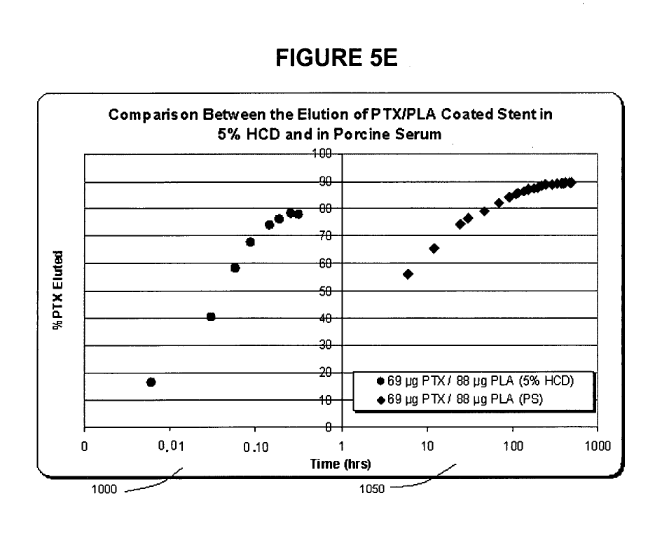

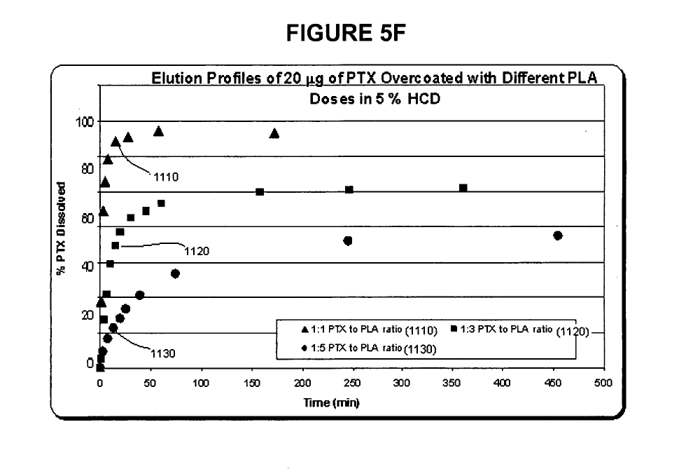

Elution profiles were obtained for different two-layer paclitaxel-eluting coatings over a layer consisting of paclitaxel deposited on the abluminal surface of a 6×20 ZILVER self-expanding vascular stents (COOK, Inc. Bloomington, Ind.). The coatings were configured to provide different sustained release rates of the therapeutic agent over a period of 21 days, or longer. FIG. 5A, FIG. 5B and FIG. 5C show elution profiles obtained from three different two-layer coatings applied to otherwise identical 6×20 mm stents (73 mm² abluminal surface area) of the same size, shape and surface area. Each drug-polymer coated stent was coated with a single (first) layer consisting of varying amounts of paclitaxel covered with a single (second) layer consisting of varying amounts of poly(D,L-lactic acid) (“PLA”) bioabsorbable elastomer. The elution of the therapeutic agent is indicated as a percentage by weight of total drug initially deposited on the stent. Various sustained paclitaxel elution profiles were achieved by varying the weight ratio of paclitaxel:PLA using coatings wherein the amounts of paclitaxel vary by almost an order of magnitude (e.g., from 5 μg to 69 μg) from coatings on numerous otherwise identical 6×20 mm ZILVER® vascular stents. Notably, comparable rates of paclitaxel release were obtained using levels of paclitaxel at low (e.g., 5 μg, or about 0.07 μg/mm² abluminal surface area) or higher (e.g., 69 μg, or about 0.95 μg/mm² abluminal surface area) amounts of paclitaxel, by varying the weight ratio of the paclitaxel to the bioabsorbable elastomer overcoat layer. Various configurations of multilayer coatings can provide sustained release of a therapeutic agent, such as paclitaxel, using dose levels of less than about 1.00 μg/mm² of the abluminal surface area of the stent.

FIG. 5A compares a slow release elution profile 510 and a faster release elution profile 550. In one embodiment, the coating is a “slow release” coating configuration that releases about 2%-10% of the therapeutic agent after 24 hours, and no more than about 50% of the therapeutic agent after about 100 hours, in an elution assay performed in a 37° C. in a porcine serum elution medium flowing at 16 mL/min. Referring again to FIG. 5A, the slow release elution profile 510 was obtained from a first stent with a second layer of 88 μg of PLA applied by an ultrasonic spray gun over a first layer of 69 μg of paclitaxel (PTX) (about a 1:1 weight ratio of paclitaxel:PLA). The paclitaxel layer was applied first as a (1.2 mM or 2.4 mM) paclitaxel solution in ethanol onto the abluminal surface of the stent and allowed to dry. Next, PLA was applied as a solution in dichloromethane to both the luminal and abluminal surfaces of the vascular stent. It is believed that the first layer (paclitaxel) is substantially free of PLA and the second layer (PLA) is substantially free of paclitaxel.

FIG. 5B shows the elution profiles for four “intermediate” release rate coating configurations. All elution profiles in FIG. 5B were obtained from coatings applied to 6×80 mm ZILVER® Nitinol vascular stents (Cook Inc., Bloomington, Ind.) using the conventional pressure gun method described with respect to elution profile 550 in FIG. 5A (i.e., without coating of the luminal surface). The coated vascular stents were placed in contact with the modified porcine serum described with respect to FIG. 4A above, at 37° C., flowing continuously at a rate of 16 mL/min. over the surface of the coated stent. The therapeutic agent for all elution profiles was paclitaxel. For comparison, the elution profile 300 from FIG. 4A and elution profile 550 from FIG. 5A are included. As described above, the elution profile 300 was obtained from a vascular stent coated with a single layer of 961 mg of paclitaxel on the abluminal surface. The remaining elution profiles 550, 610 and 620 were obtained from vascular stent having a first layer of varying amounts of paclitaxel between the abluminal surface of the vascular stent and a second layer of varying amounts of poly(D,L)-lactic acid over the first layer. Each layer was applied separately from a pressure gun by spraying a solution of paclitaxel in ethanol, followed by a solution of poly(D,L)-lactic acid in dichloromethane. The elution profiles 300, 550, 610 and 620 were all obtained as described with respect to FIG. 4A above, using a modified procine serum elution medium at 37° C. and flowing at 16 mL/min, and detecting the presence of paclitaxel in the elution medium using UV-Vis spectroscopy.

Increasing the amount of bioabsorbable elasomer in the second layer while keeping the amount of paclitaxel in the first layer constant can slow the comparative elution rate, as seen by comparing elution profile 550 and elution profile 610 in FIG. 5B. The stent from which elution profile 550 was obtained is described with respect to FIG. 5A (a paclitaxel:PLA weight ratio of about 1:1.25). The elution profile 610 was obtained from a coated vascular stent that is identical to the vascular stent described with respect to FIG. 5A, except that the second layer contains more PLA (173 μg poly(D,L)-lactic acid instead of 88 μg), with a paclitaxel:PLA weight ratio of about 1:2.50. The elution profile 610 shows a slower rate of paclitaxel elution than the elution profile 550, with about 60% of the paclitaxel dissolved after 100 hours (about 4 days) compared to about 80% paclitaxel elution in the elution profile 550.

Decreasing the amount of therapeutic agent in the first layer while keeping the amount of bioabsorbable elastomer in the second layer constant can also slow the comparative elution rate, as seen by comparing elution profile 610 (a paclitaxel:PLA weight ratio of about 1:2.5) and elution profile 620 in FIG. 5B. The stent from which elution profile 610 was obtained is described above. The elution profile 620 was obtained from a coated vascular stent that is identical to the vascular stent described with respect to elution profile 610, except that the first layer contains less paclitaxel (5 μg paclitaxel instead of 69 μg) (a paclitaxel:PLA weight ratio of about 1:15 instead of about 1:2.5). The elution profile 620 shows a slower rate of paclitaxel elution than the elution profile 620, with about 50% of the paclitaxel dissolved after 100 hours (about 4 days) compared to about 60% paclitaxel elution in the elution profile 610.

FIG. 5C shows the elution profiles for six coating configurations. All elution profiles in FIG. 5C were obtained from coatings applied to 6×20 mm ZILVER® Nitinol vascular stents (Cook Inc., Bloomington, Ind.) using the ultrasonic spray gun method used to obtain the elution profile 510 in FIG. 5A (i.e., coating of the luminal surface with PLA but not paclitaxel). The coated vascular stents were placed in contact with the modified porcine serum described with respect to FIG. 4A above, at 37° C., flowing continuously at a rate of 16 mL/min. over the surface of the coated stent. The therapeutic agent for all elution profiles was paclitaxel. FIG. 5C shows a graph of drug elution from a two-layer paclitaxel-PLA coated stent, where the PLA was applied over a layer of paclitaxel using an ultrasonic nozzle coating process. The elution profile 510 from FIG. 5A is included for comparison. The elution profile 510 had the a rate of paclitaxel elution similar to elution profile 710, and was obtained from a stent coated with 20 μg of paclitaxel in a first layer covered with 185 μg of PLA in a second layer (a paclitaxel:PLA weight ratio of about 1:9). Less than 20% of the paclitaxel in the first elution profile 510 dissolved after about 4 days. The elution profile 710 was obtained from a stent coated with 67 μg of paclitaxel in a first layer covered with 265 μg of PLA in a second layer (a paclitaxel:PLA weight ratio of about 1:4). The elution profile 720 was obtained from a stent coated with 14 μg of paclitaxel in a first layer covered with 199 μg of PLA in a second layer (a paclitaxel:PLA weight ratio of about 1:14). The elution profile 720 showed a more rapid rate of paclitaxel elution than the elution profile 710, with approximately 25% of the paclitaxel in the elution profile 720 dissolved after about 4 days. The elution profile 730 showed a more rapid rate of paclitaxel elution than elution profile 720, and was obtained from a stent coated with 62 μg of paclitaxel in a first layer covered with 86 μg of PLA in a second layer (a paclitaxel:PLA weight ratio of about 1:1.3). The elution profile 740 showed a comparable rapid rate of paclitaxel elution to elution profile 730, and was obtained from a stent coated with 7 μg of paclitaxel in a first layer covered with 172 μg of PLA in a second layer (a paclitaxel:PLA weight ratio of about 1:25). Approximately 25-30% of the paclitaxel in the elution profiles 730 and 740 dissolved after about 4 days. The elution profile 750 showed a more rapid rate of paclitaxel elution than elution profiles 740 or 730, and was obtained from a stent coated with 7 μg of paclitaxel in a first layer covered with 123 μg of PLA in a second layer (a paclitaxel:PLA weight ratio of about 1:18). Approximately 35-40% of the paclitaxel in the elution profile 750 dissolved after about 4 days. Increasing the ratio of PLA to paclitaxel generally slowed the rate of elution. FIG. 5D shows the elution profiles in porcine serum at 37 C for six different two-layer coatings formed from varying amounts of a PLA layer over a paclitaxel layer on the abluminal surface of a 6×20 ZILVER stent. The elution profile in FIG. 5D show elution profiles that vary as a function of the amount of PLA and paclitaxel in a manner similar to the elution profiles in FIG. 5C.

For therapeutic agents that are soluble in a cyclodextrin solution, such as taxane therapeutic agents, elution profiles may also be obtained by contacting a coated medical device with an elution medium comprising a cyclodextrin. A cyclodextrin is a cyclic oligosaccharide formed from covalently-linked glucopyranose rings defining an internal cavity. The diameter of the internal axial cavity of cyclodextrins increases with the number of glucopyranose units in the ring. The size of the glucopyranose ring can be selected to provide an axial cavity selected to match the molecular dimensions of a taxane therapeutic agent. The cyclodextrin is preferably a modified β-cyclodextrin, such as Heptakis-(2,6-di-O-methyl)-β-cyclodextrin (HCD). Suitable cyclodedtrin molecules include other β-cyclodextrin molecules, as well as γ-cyclodextrin structures.

The elution medium comprising a cyclodextrin can dissolve a taxane therapeutic agent so as to elute the taxane therapeutic agent from a medical device coating over a desired time interval, typically about 24 hours or less. Preferably, the cyclodextrin elution medium is formulated to provide distinguishable elution rates for different coating configurations, such as different solid forms of a taxane therapeutic agent in the coating, or different types or amounts of polymers incorporated with the taxane therapeutic agent within a coating.

An elution medium comprising a suitable cyclodextrin may be useful in providing an elution profile indicative of the composition or configuration of a medical device coating comprising a taxane therapeutic agent, and useful to provide lot release data pertaining to the coating of the medical device. For example, the elution profile of a medical device coating formed from a solvated solid form of a taxane therapeutic agent measured in a cyclodextrin elution medium typically provides a distinguishably slower rate of elution than a medical device coating formed from an amorphous solid form of the taxane therapeutic agent in the same elution medium. Similarly, the elution profile of a coating comprising both a taxane therapeutic agent and differing amounts of a biodegradable elastomer, such as poly(lactic acid), can be distinguished based on the elution profiles in a cyclodextrin elution medium. Obtaining an elution profile by contacting a taxane-coated medical device with an elution medium comprising a suitable cyclodextrin provides a method for obtaining lot release data indicative of differences in coating configuration that are distinguishable based on solubility of the taxane therapeutic agent in the cyclodextrin.

FIG. 5E and FIG. 5F are elution profiles showing the elution rates of comparable medical device coatings comprising paclitaxel and a biodegradable polymer in two different solvents (porcine serum and β-cyclodextrin). To obtain the data for both FIG. 5E and FIG. 5F, the amount of paclitaxel eluted was determined by monitoring the characteristic peak of paclitaxel at 227 nm by UV detection within the elution media after contacting the medical device coating, as described above.

FIG. 5E shows a first elution profile (1000) and a second elution profile (1050) both obtained from two substantially identical coated vascular stents, each comprising a two-layer coating with a first layer of paclitaxel deposited on the outer surface of the stent and a second layer of PLA deposited over and enclosing the first layer of paclitaxel. The first coating layer on each coated stent included a total of 69 μg of paclitaxel, covered by a total of 88 μg of PLA. The first elution profile 1000 was obtained by contacting the first coated stent with a continuous flow of an aqueous elution medium with 5% HCD, while the second elution profile 1050 was obtained by placing the second coated stent in a continuous flow of porcine serum. The coating eluted much more rapidly in the HCD cyclodextrin elution medium than the porcine serum elution medium. In the first elution profile 1000, about 70% of the paclitaxel eluted after about 0.1 hours (6 minutes), and about 80% of the coating eluted within about 1 hour. In contrast, in the second elution profile 1050, less than 60% of the paclitaxel eluted after about 6 hours, less than 70% after about 10 hours, and nearly 100 hours were required to elute 90% of the paclitaxel. Accordingly, the use of porcine serum as an elution medium can require extended testing periods to ascertain the elution profile of paclitaxel from a coating comprising a polymer and paclitaxel, while substantially less time may be required to obtain comparable data when using a cyclodextrin elution medium.

FIG. 5F shows a set of three elution profiles 1100 obtained from substantially identical coated vascular stents having similar two-layer PLA-paclitaxel coatings, but differing in the ratio of PLA to paclitaxel in the coating. All three coatings have a first layer of 20 μg paclitaxel applied to the exterior surface of substantially identical vascular stents, and a second layer of PLA applied over and enclosing the first layer. The coatings differed in the amount of PLA in the second layer. All three elution profiles 1100 were obtained by placing the coated stents in a continuous flow of an aqueous solution of 5% HCD cyclodextrin elution medium. The first elution profile 1110 was obtained from a coating having 20 μg of PLA (a paclitaxel:PLA mass ratio of 1:1) (shown as triangular data points), and eluted most rapidly of the three coatings. The second elution profile 1120 was obtained from a coating having 60 μg of PLA (a paclitaxel:PLA mass ratio of 1:3) (shown as square data points), and eluted more slowly than the coated stent of the first elution profile 1110. The third elution profile 1140 was obtained from a coating having 100 μg of PLA (a paclitaxel:PLA mass ratio of 1:5) (shown as circular data points), and eluted the most slowly of the three elution profiles 1100.

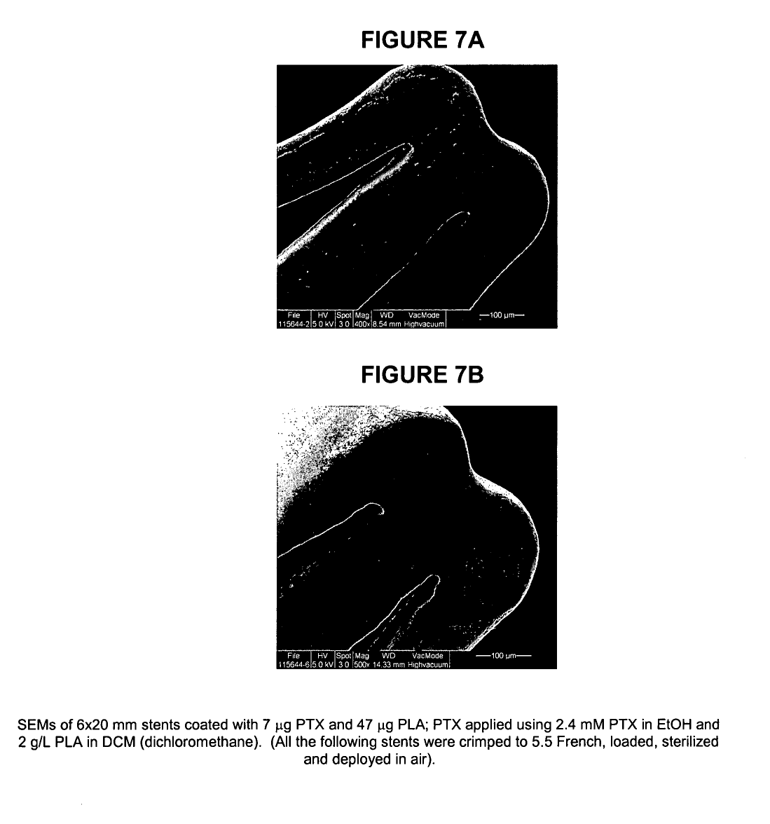

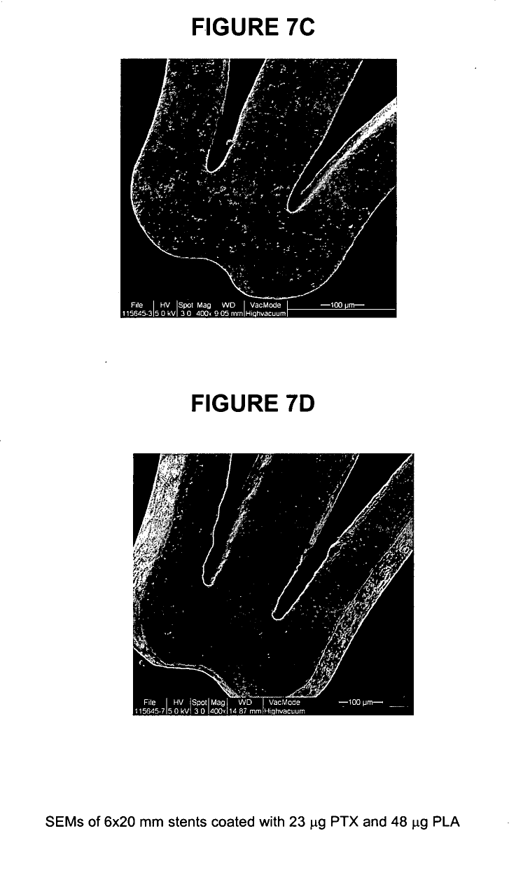

Increasing the amount of PLA relative to the amount of paclitaxel decreased the elution rate of the paclitaxel in cyclodextrin elution medium. Referring to Example 6 below, elution of similar two-layer coatings of PLA over paclitaxel in porcine serum also demonstrate an increase in the elution time of paclitaxel as the amount of PLA is increased. The coatings eluted in Example 6, like the second elution profile 1050 in FIG. 7, also required extended times of over 100 hours to elute up to about 70% to 90% of the paclitaxel, depending on the amount of PLA. Such lengthy elution times can be disadvantageous in obtaining lot release data.

Biodegradable Elastomers

The bioabsorbable elastomer is preferably a polymer selected to provide a mechanically stable coating layer that readily recovers from deformation of the medical device without undesirable levels of irritation to surrounding tissue upon implantation. The bioabsorbable elastomer can include a hydrogel, an elastin-like peptide, a polyhydroxyalkanoates (PHA), polyhydroxybutyrate compounds, or combinations thereof. The bioabsorbable elastomer can be selected based on various design criteria, including the desired rate of release of the therapeutic agent and the degradation mechanism. In some embodiments, the bioabsorbable elastomer comprises one or more hydrolyzable chemical bonds, such as an ester, a desired degree of crosslinking, a degradation mechanism with minimal heterogeneous degradation, and nontoxic monomers.

The bioabsorbable elastomer may be a polyhydroxyalkanoate compound, a hydrogel, poly(glycerol-sebacate) or an elastin-like peptide. Desirably, the bioabsorbable elastomer includes a polyhydroxyalkanoate bioabsorbable polymer such as polylactic acid (poly lactide), polyglycolic acid (poly glycolide), polylactic glycolic acid (poly lactide-co-glycolide), poly4-hydroxybutyrate, or a combination of any of these. Preferably, the therapeutic agent is initially enclosed by the coating or other portions of the medical device, and does not form a portion of the external surface area of the medical device prior to release of the therapeutic agent.

Desirably, the bioabsorbable elastomer comprises a poly-α-hydroxy acid, such as polylactic acid (PLA). PLA can be a mixture of enantiomers typically referred to as poly-D,L-lactic acid. Alternatively, the bioabsorbable elastomer is poly-L(+)-lactic acid (PLLA) or pol-D(−)-lactic acid (PDLA), which differ from each other in their rate of biodegradation. PLLA is semicrystalline. In contrast, PDLA is amorphous, which can promote the homogeneous dispersion of an active species. Unless otherwise specified, recitation of “PLA” herein refers to a bioabsorbable polymer selected from the group consisting of: PLA, PLLA and PDLA. Preferably, the molecular weight of the bioabsorbable elastomer is about 50-500 kDa, more preferably about 60-250 kDa, and most preferably about 75-120 kDa.

The bioabsorbable elastomer can also desirably comprise polyglycolic acid (PGA). Polyglycolic acid is a simple aliphatic polyester that has a semi-crystalline structure, fully degrades in 3 months, and can undergo strength loss within about 1 month after implantation in the body. Compared with PLA, PGA is a stronger acid and is more hydrophilic, and thus more susceptible to hydrolysis. PLA is generally more hydrophobic than PGA, and undergoes a complete mass loss in 1 to 2 years.

The bioabsorbable elastomer can also be a polylactic glycolic acid (PLGA), or other copolymers of PLA and PGA. The properties of the copolymers can be controlled by varying the ratio of PLA to PGA. For example, copolymers with high PLA to PGA ratios generally degrade slower than those with high PGA to PLA ratios. PLGA degrades slightly faster than PLA. The process of lactic acid hydrolysis can be slower than for the glycolic acid units of the PLGA co-polymer. Therefore, by increasing the PLA:PGA ratio in a PLGA co-polymer generally results in a slower rate of in vivo bioabsorption of a PLGA polymer.

A summary of the properties of some desirable bioabsorbable elastomer polymers are shown below in Table 2.

TABLE 2

| | | Degradation Rate | | |
|---|---|---|---|---|
| | | (depends on molecular | | |
| Polymer | Crystallinity | weight of polymer) | | Typical Applications |
| PGA | High | 2-3 | months | Suture, soft anaplerosis |
| | Crystallinity | | | |
| PLLA | Semi- | >2 | years | Fracture fixation, |
| | crystalline | | | ligament |
| PDLA | Amorphous | 12-16 | months | Drug delivery system |
| PLGA | Amorphous | 1-6 | months | Suture, fracture fixation, |

| | |
|---|---|
| (depends on ratio of | oral implant, drug |
| LA to GA | delivery |

Cross-linked polymers of glycerol and sebacic acid can also be used as the bioabsorbable elastomer, such as a poly4-hydroxybutyrate (P4HB) or poly(glycerol-sibacate) (PGS). PGS can be prepared by the polycondensation of glycerol and sebacic acid to yield an elastomer. PGS can be formed with any suitable ratio of glycerol:sebacic acid. Preferably, the bioabsorbable elastomer is a PGS with 1:1 glycerol:sebacic acid ratio, which is largely insoluble in water and swells about 2% after soaking in water for 24 hours, can have a cross-linking density of about 38 mol/m³ and two DSC melting temperatures at 5.23° C. and 37.62° C. Accordingly, the 1:1 PGS polymer is completely amorphous at 37° C. within the body. The preparation and characterization of a 1:1 glycerol:sebacic acid PGS bioabsorbable elastomer is described in Y. Wang et al., “A tough biodegradable elastomer,” Nature Biotechnology, 20, 602-606 (2002), which is incorporated herein by reference. Briefly, the 1:1 PGS can be prepared in an uncrosslinked prepolymer that can be melted into a liquid and dissolved in common organic solvents including 1,3-dioxolane, tetrahydrofuran, ethanol, isopropanol and N,N-dimethylformamide. A mixture of NaCl particles and an anhydrous 1,3-dioxolane prepolymer can be poured into a PTFE mold. The polymer can be cured in the mold in a vacuum oven at 120° C. and 100 mtorr, and a porous scaffold can be obtained after salt leaching with deionized water. Desirably, the PGS bioabsorbable elastomer has a strain to failure property similar to that of arteries and veins (e.g., up to about 260%) and larger than tendons (up to about 18%). Furthermore, the weight of PGS can remain substantially unchanged after soaking 24 hours in an aqueous environment, and the mechanical properties can remain largely unchanged compared to the dry polymer. Y. Wang et al. reported that 1:1 PGS degrades about 17% after 60 days in PBS solution at 37° C., as measured by change in weight; subcutaneous implantation of the 1:1 PGS in rats lead to complete absorption of the polymer in 60 days (Y. Wang et al., “A tough biodegradable elastomer,” Nature Biotechnology, 20, 602-606 (2002)). Data indicated that mechanical strength of the 1:1 PGS decreases linearly with mass loss, suggesting a surface erosion mechanism (Y. Wang et al., “A tough biodegradable elastomer,” Nature Biotechnology, 20, 602-606 (2002)).

Alternative ratios of glycerol:sebacic acid can also be prepared, including a 2:3 PGS ratio polymer described by M. Nagata et al., “Synthesis, characterization, and enzymatic degradation of network aliphatic copolyesters,” J. Polym. Sci. Part A: Polym. Chem., 37, 2005-2011.

Desirably, polymers used in coating layers are not waxy or tacky, adequately adhere to the surface of the medical device, and deform readily after it is adhered to the device. The molecular weight of the polymer(s) should be high enough to provide sufficient toughness so that the polymers will not be rubbed off during sterilization, handling, or deployment of the medical device and will not crack when the device is expanded. Exemplary polymer systems that may also be used in one or more coating layers include polymers that are biocompatible and minimize irritation when the medical device is implanted. The polymer may be either a biostable or a bioabsorbable polymer, depending on the desired rate of release or the desired degree of polymer stability. A bioabsorbable polymer may be preferred in certain embodiments because, unlike a biostable polymer, it will not be present long after implantation to cause any adverse, chronic local response. The properties of any mixture of polymers depend primarily on thermodynamic miscibility. If the polymers are immiscible, the properties will depend not only on the properties of each component, but also on the morphology and adhesion between the phases.

Coating Methods

In a second embodiment, methods of coating a surface of an implantable medical device are provided. The coating may be applied to a surface of an implantable medical device by any suitable method. Coating layers may be applied in sequential fashion to the surface of the medical device. Preferably, a layer comprising a therapeutic agent is first applied over the surface of the implantable medical device, and another layer comprising a biodegradable elastomer is applied over the therapeutic agent. The coating layers can be deposited on the surface of an implantable medical device or be locally deposited within holes or wells in the surface of the medical device. Three preferred methods for applying coating layers are described herein: (1) spray gun coating, (2) ultrasonic spray coating and (3) electrostatic spray coating.

In all three methods, a coating layer comprising a therapeutic agent can be formed by applying a first solution of the therapeutic agent to the surface of the medical device. Preferably, the first solution consists essentially of the therapeutic agent and a volatile solvent, and does not contain the bioabsorbable elastomer. Desirably, the therapeutic agent is paclitaxel and the solvent is ethanol or methanol. Desirably, a solution of about 0.5-5.0 mM paclitaxel in ethanol may used, preferably solutions of 0.7 mM, 1.2 mM paclitaxel in ethanol. Other therapeutic agents and solvents may also be used in solutions at concentrations permitting desirable deposition rates forming coatings with desired durability.

After the application of the therapeutic agent, another layer comprising a bioabsorbable elastomer material can be dissolved in a solvent and then sprayed onto a layer of therapeutic agent that was previously deposited on the medical device. Desirably, the polymer is PLA and the solvent is dichloromethane. More desirably, about 0.1-7.0 g/L of PLA in dichloromethane is used. Even more desirably, about 2.5-6.5 g/L and most desirably 5.0 g/L of PLA in dichloromethane is used.

Each coating layer is preferably separately applied using an ultrasonic nozzle spray coating technique employing ultrasound to atomize the spray solution, to provide a smooth and uniform polymer coating. Preferably, the polymer coating is applied from an ultrasonic nozzle. A solution of about 2-4 g/L of a bioabsorbable elastomer such as PLA in a suitable solvent such as dichloromethane can be applied using an ultrasonic nozzle. Ultrasonic nozzles can be configured such that excitation of the piezoelectric crystals creates a transverse standing wave along the length of the nozzle. The ultrasonic energy originating from the crystals located in the large diameter of the nozzle body undergoes a step transition and amplification as the standing wave as it traverses the length of the nozzle. The ultrasonic nozzle can be designed so that a nodal plane is located between the crystals. For ultrasonic energy to be effective for atomization, the atomizing surface (nozzle tip) is preferably located at an anti-node, where the vibration amplitude is greatest. To accomplish this, the nozzle's length must be a multiple of a half-wavelength. Since wavelength is dependent upon operating frequency, nozzle dimensions can be related to operational frequency. In general, high frequency nozzles are smaller, create smaller drops, and consequently have smaller maximum flow capacity than nozzles that operate at lower frequencies. The ultrasonic nozzle can be operated at any suitable frequency, including 24 kHz, 35 kHz, 48 kHz, 60 kHz, 120 kHz or higher. Preferably, a frequency of 60-120 kHz or higher is used to atomize the solution of the bioabsorbable elastomer to the greatest possible extent so as to promote the formation of a smooth, uniform coating. Power can be controlled by adjusting the output level on the power supply. The nozzle power can be set at any suitable level, but is preferably about 0.9-1.2 W and more preferably about 1.0-1.1 W. The nozzle body can be fabricated from any suitable material, including titanium because of its good acoustical properties, high tensile strength, and excellent corrosion resistance. Liquid introduced onto the atomizing surface through a large, non-clogging feed tube running the length of the nozzle absorbs some of the vibrational energy, setting up wave motion in the liquid on the surface. For the liquid to atomize, the vibrational amplitude of the atomizing surface can be miantained within a band of input power to produce the nozzle's characteristic fine, low velocity mist. Since the atomization mechanism relies only on liquid being introduced onto the atomizing surface, the rate at which liquid is atomized depends largely on the rate at which it is delivered to the surface. Therefore, an ultrasonic nozzle can have a wide flow rate range. The maximum flow rate and median drop diameter corresponding to particular nozzle designs can be selected as design parameters by one skilled in the art. Preferably, the flow rate is between about 0.01-2.00 mL/min, more preferably between about 0.05-1.00 and most preferably between about 0.05-0.07 mL/min. Preferred coating parameters for USD using a Sono-tek Model 8700-60 ultrasonic nozzle are provided in Table 3 below:

TABLE 3
Ultrasonic Spray Deposition Parameters
for Sono-tek Model 8700-60

| | Coating | Rotation | Nozzle | | |
|---|---|---|---|---|---|
| Flow rate | velocity | Speed | Power | Process | Distance |
| (mL/min) | (in/sec) | (rpm) | (watts) | Gas (psi) | (mm) |
| 0.01-2 | 0.01-0.5 | 30-150 | 0.9-1.2 | 0.1-2.5 | 1-25 |

Alternatively, the therapeutic agent(s) and bioabsorbable elastomer can be dissolved in a solvent(s) and sprayed onto the medical device using a conventional spray gun such as a spray gun manufactured by Badger (Model No. 200), an electrostatic spray gun, or most preferably an ultrasonic nozzle spray gun. Medical device coatings comprising a taxane therapeutic agent may be applied to a surface of a medical device using a spray gun. The surface of the medical device can be bare, surface modified, or a primer coating previously applied to the medical device. Preferably, the coating applied to the surface consists essentially of the taxane therapeutic agent, and is substantially free of polymers or other materials. The taxane therapeutic agents, and optionally a polymer, can be dissolved in a solvent(s) and sprayed onto the medical device under a fume hood using a conventional spray gun, such as a spray gun manufactured by Badger (Model No. 200), or a 780 series spray dispense valve (EFD, East Providence, RI). Alignment of the spray gun and stent may be achieved with the use of a laser beam, which may be used as a guide when passing the spray gun over the medical device(s) being coated.

Desirably, the therapeutic agent is paclitaxel and the solvent is ethanol or methanol. Desirably, a solution of paclitaxel in ethanol described above is used. The distance between the spray nozzle and the nozzle size can be selected depending on parameters apparent to one of ordinary skill in the art, including the area being coated, the desired thickness of the coating and the rate of deposition. Any suitable distance and nozzle size can be selected. For example, for coating an 80 mm long stent, a distance of between about 1-7 inches between the nozzle and stent is preferred, depending on the size of the spray pattern desired. The nozzle diameter can be, for example, between about 0.014-inch to about 0.046-inch.

Varying parameters in the spray coating process can result in different solid forms of the taxane therapeutic agent in a deposited coating. Spray coating parameters such as solvent system, fluid pressure (i.e., tank pressure), atomization pressure, ambient temperature and humidity. The solvent is desirably volatile enough to be readily removed from the coating during or after the spray coating process, and is preferably selected from the solvents discussed with respect to the first embodiment for each solid form of a taxane therapeutic agent.

Methods of coating amorphous taxane therapeutic agents using a 780S-SS spray dispense valve (EFD, East Providence, RI) can comprise the steps of: dissolving solid paclitaxel in ethanol to form a solution, and spraying the solution onto a medical device with an atomization pressure of about 5-10 psi in an environment having a relative humidity of 30% or lower. Preferably, the spraying step is performed at a temperature of between about 65° F. and 75° F., and with a fluid pressure of between about 1.00 and 5.00 psi.

One or more coating layers may also be applied using an electrostatic spray deposition (ESD) process. This process is especially desirable when the therapeutic agent is hydrophilic. The ESD process generally depends on the principle that a charged particle is attracted towards a grounded target. The solution that is to be deposited on the target is typically charged to several thousand volts (typically negative) and held at ground potential. The charge of the solution is generally great enough to cause the solution to jump across an air gap of several inches before landing on the target. As the solution is in transit towards the target, it fans out in a conical pattern which aids in a more uniform coating. In addition to the conical spray shape, the electrons are further attracted towards the metal portions of the target, rather than towards the non conductive base the target is mounted on, leaving the coating mainly on the target only.

Generally, the ESD method allows for control of the coating composition and surface morphology of the deposited coating. In particular, the morphology of the deposited coating may be controlled by appropriate selection of the ESD parameters, as set forth in WO 03/006180 (Electrostatic Spray Deposition (ESD) of biocompatible coatings on Metallic Substrates), incorporated herein by reference. For example, a coating having a uniform thickness and grain size, as well as a smooth surface, may be obtained by controlling deposition conditions such as deposition temperature, spraying rate, precursor solution, and bias voltage between the spray nozzle and the medical device being coated. The deposition of porous coatings is also possible with the ESD method.

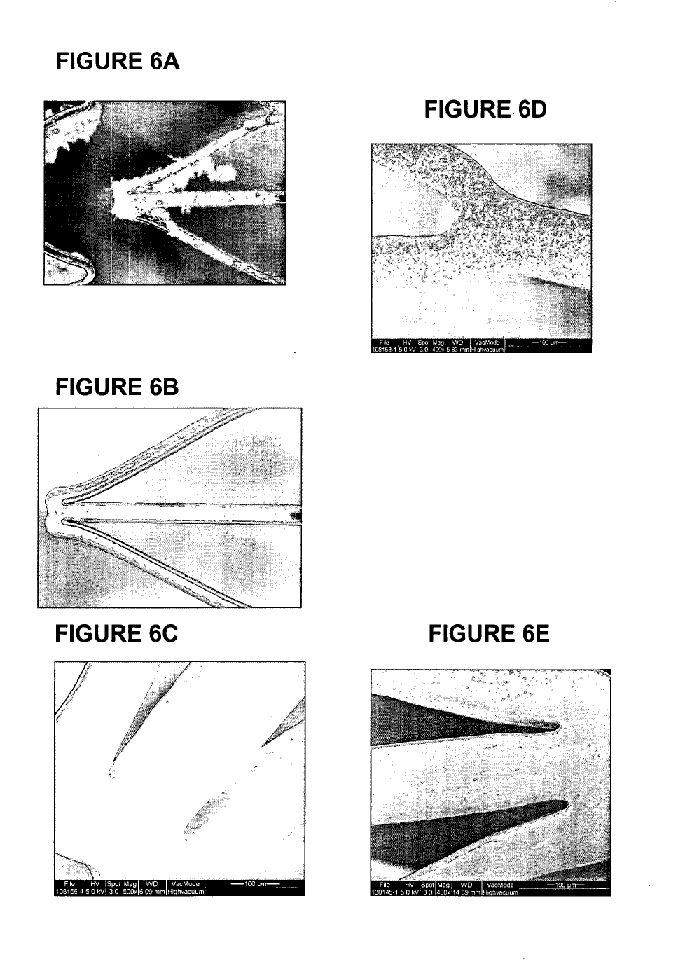

The bioabsorbable elastomer (such as PLA) for spraying onto the medical device using the ESD method, is preferably dissolved in a solvent mixture comprising a mixture of dichloromethane:methanol in a 1:2 (+/−10%) ratio by volume. For example, the solvent mixture can comprise about 50-80% methanol and about 20-50% dichloromethane (by volume). More desirably, the mixture is about 65-75% methanol and about 25-40% dichloromethane (by volume). Even more desirably, the mixture is about 70% methanol and about 30% dichloromethane (by volume). It is believed that the addition of methanol to dichloromethane increases the polarity of the solvent solution, thereby providing a fine spray that is ideal for use in an electrostatic coating process. This solvent combination may provide a smooth, uniform bioabsorbable elastomer coating when applied by spraying. FIG. 6A shows an optical micrograph of a first PLA coating applied with a dichloromethane solvent using an ESD coating process. The width of the stent struts is approximately 150 μm-200 μm. The first PLA coating was highly fragmented and unevenly distributed on the surface of the stent. FIG. 6B shows an optical micrograph of a second PLA coating applied by ESD with a solvent mixture of dichloromethane and methanol in a 1:2 ratio by volume. The image has approximately the same scale as FIG. 6A. The second PLA coating was smooth, highly uniform and has an estimated surface roughness of about 0.25 to 2.00 microns.

Coating Uniformity and Durability

The coatings are also preferably sufficiently durable to withstand percutaneous transcatheter deployment in a radially compressed state, which can include resistance to flaking, chipping or crumbling of the coating during crimping onto a catheter delivery system.

Desirably, coatings have sufficient durability to retain a desired amount of a therapeutic agent after manipulations typically associated with the manufacture and delivery of the medical devices to a desired point of treatment, and to function to release the therapeutic agent at the point of treatment at a desired rate. Durable coatings on medical device preferably resist flaking, pitting or delamination as a result of physical abrasion, compression, flexion, vibration, fluid contact, and fluid shear. For implantable vascular stents, coatings are desirably durable enough to maintain a substantially uniform coating during sterilization, radial compression by crimping onto a delivery catheter, and radial expansion within a blood vessel at a point of treatment.

The durability of a coating can be evaluated by weighing the medical device a first time immediately after coating, subjecting the coated medical device to physical forces typical of the manufacture and delivery process for an intended use (e.g., crimping, freezing, sterilization and the like), and then weighing the coated medical device a second time. A loss in weight between the first weighing and the second weighing could indicate the loss of portions of the coating to flaking or delamination. Preferably, durable coatings for implantable vascular stents loose no more than about 10 μg or about 20% of the coating weight or less before and after crimping. A durable coating preferably loses less than about 15%, more preferably between about 0-10%, most preferably between about 0% and 5% of the weight of the coating during the crimping process. Durable coatings are also substantially free of “webbing,” or coating deposited over interstitial spaces between portions of a medical device.

The durability of comparable two-layer coatings having a layer of PLA deposited over a layer of paclitaxel on the albuminal surface of a 6×20 ZILVER stent (Cook Inc., Bloomington, Ind.). Six stents were coated using an ultrasonic spray gun, and ten stents were coated using a standard spray gun. The coating layers were coated from the same solutions (a paclitaxel-ethanol solution for the first layer, and a PLA-dichloromethane solution for the second layer). The coated stents were crimped to a diameter of 5.5 French (about 1.8 mm) and sterilized by a standard ethylene oxide process. The sterilization process included subjecting the coated stents to temperatures of about 40 C and humidity levels of over 90%, followed contact with ethylene oxide at about 575 mg/L for a suitable period of time to perform the sterilization. After sterilization, the coated stents were again measured, and the weight loss of the coating during the crimping and sterilization processes was calculated.

The results from the durability measurements for the conventional spray coated stent coatings (Table 4) show a loss of 0-17%, with most stents losing 8% or more of the coating due to crimping and sterilization.

TABLE 4
Durability: Spray Gun Coating

| | | | Average Weight | | |
|---|---|---|---|---|---|
| | | Weight | After | | |
| | Bare Stent | After | Deployment & | | |
| | Weight | PTX & | Sterilization | Δ | % Loss/ |
| Stent | (μg) | PLA (μg) | (μg) | Weight | Gain |
| 1 | 92825 | 92875 | 92879 | 4 | 8 |
| 2 | 92758 | 92805 | 92809 | 4 | 8 |
| 3 | 89947 | 90001 | 90011 | 9 | 17 |
| 4 | 91171 | 91496 | 91506 | 9 | 3 |
| 5 | 92736 | 92801 | 92810 | 8 | 13 |
| 6 | 92927 | 93115 | 93115 | 1 | 0 |

In contrast, the results from the durability measurements for the conventional spray coated stent coatings (Table 5) show a loss of 0-4%, with many of the stents not having any measurable coating loss due to crimping and sterilization. Accordingly, the coatings are preferably applied by ultrasonic spray deposition.

TABLE 5
Durability: Ultrasonic Coating,

| | | Weight | Wt. after | | |
|---|---|---|---|---|---|
| | Naked | after | Sterili- | | |
| | Weight | PTX & | zation | Δ | % Loss/ |
| Stent # | (μg) | PLA (μg) | (μg) | (μg) | Gain |
| 7 | 87738 | 87901 | 87901 | 0 | 0 |
| 8 | 91411 | 91580 | 91580 | 0 | 0 |
| 9 | 88843 | 89008 | 89008 | 0 | 0 |
| 10 | 90727 | 90821 | 90825 | 4 | 4 |
| 11 | 86840 | 86929 | 86926 | −3 | N/A |
| 12 | 89298 | 89398 | 89398 | 0 | 0 |
| 13 | 88781 | 88902 | 88904 | 2 | 2 |
| 14 | 89707 | 89827 | 89826 | −1 | N/A |
| 15 | 89288 | 89554 | 89558 | 4 | 2 |
| 16 | 91630 | 91858 | 91860 | 2 | 1 |

Preferably, the coatings have a substantially uniform surface, without cracking or pitting. Desirably, coatings have a surface that retains surface uniformity and integrity upon sterilization and crimping. Various coating methods can be used to produce suitably smooth and durable coatings. Preferably, the top layer of a coating comprises a bioabsorbable elastomer. Substantially uniform and durable coatings can be deposited by spraying a solution of a therapeutic agent or bioabsorbable elastomer onto the abluminal surface of a medical device using conventional pressure gun, electrostatic spray gun and ultrasonic spray gun. The uniformity of a coating can be evaluated from optical and SEM images of the surfaces.

FIG. 6C is a scanning electron microscope (SEM) image of the surface (500×) of a durable and uniform coating on a radially expandable vascular stent after sterilization, crimping and deployment of the vascular stent. Visible in FIG. 6C is the top layer of a two layer coating applied over an underlying layer of paclitaxel. To form the coating of FIG. 6C, a 2.4 mM solution of paclitaxel in ethanol was spray coated onto the abluminal surface of a nitinol vascular stent and allowed to dry until the ethanol evaporated, leaving about 25 μg of paclitaxel on the surface of the vascular stent. Then, a layer of 132 μg of PLA was applied by spraying a 4.0 g/L solution of PLA in dichloromethane onto to paclitaxel layer. The highly uniform upper layer in FIG. 6C is magnified about 500×, and was obtained after manipulations that are typical in the PCTA delivery process (sterilization, crimping to a radially compressed configuration and expansion).

FIG. 6D is an SEM image of the surface of a durable and uniform coating on a radially expandable vascular stent after crimping of the vascular stent. Visible in FIG. 6D is the top layer of a two layer coating applied over an underlying layer of paclitaxel. To form the coating of FIG. 6D, a 2.4 mM solution of paclitaxel in ethanol was spray coated onto the abluminal surface of a nitinol vascular stent and allowed to dry until the ethanol evaporated, leaving about 25 μg of paclitaxel on the surface of the vascular stent. Then, a layer of 132 μg of PLA was applied by spraying a 2.14 g/L solution of PLA in a 2.5:1 methanol:dichloromethane solvent onto to paclitaxel layer at a coating rate of about 4 mL/hr for 2 minutes. The uniform upper layer in FIG. 6D is magnified about 400×, and was obtained after crimping, but without sterilization. The surface of the coated stent in FIG. 6D is slightly rougher than the stent shown in FIG. 6C, with a roughened surface morphology on approximately a 1-5 μm scale. By comparison, FIG. 6B shows a substantially uniform coating of PLA applied over a layer of paclitaxel by electrostatic spray coating of the PLA, as described above, with a smoother surface.

FIG. 6E is an SEM image of the surface of a durable and uniform coating on a radially expandable vascular stent after sterilization, crimping and deployment of the vascular stent. At 300× magnification, FIG. 6E shows the top layer of a two layer coating applied over an underlying layer of paclitaxel. To form the two layer coating shown in FIG. 6E, a 2.4 mM solution of paclitaxel in ethanol was spray coated onto the abluminal surface of a nitinol vascular stent using a conventional pressure gun and allowed to dry until the ethanol evaporated, leaving about 49 μg of paclitaxel on the surface of the vascular stent. Then, a layer of 203 μg of PLA was applied by spraying a 4.0 g/L solution of PLA in dichloromethane onto to paclitaxel layer using an ultrasonic spray nozzle. The ultrasonic spray nozzle was operated at a coating velocity of 0.05 in/sec, a nozzle power of 1.1 Watts, a flow rate of 0.06 mL/min, an air shroud pressure of 0.5 psi, and a 4 mm distance from the nozzle to the abluminal surface of the stent. The stent was rotated during the coating process and the nozzle was rastered longitudinally to provide a substantially uniform coating. The highly uniform upper layer in FIG. 6E was obtained after manipulations that are typical in the PCTA delivery process (sterilization, crimping to a radially compressed configuration and expansion). The surface of the covering layer desirably has a surface roughness less than about 10 micrometers, such as between about 0.1 μm and about 5.0 μm.

Optionally, one or more primer layers may be applied between the surface of the medical device and a therapeutic agent to adhere the therapeutic agent to the surface or enhance the durability of the coating. A primer layer, or adhesion promotion layer, may be used with the present invention. This layer may include, for example, silane, acrylate polymer/copolymer, acrylate carboxyl and/or hydroxyl copolymer, polyvinylpyrrolidone/vinylacetate copolymer, olefin acrylic acid copolymer, ethylene acrylic acid copolymer, epoxy polymer, polyethylene glycol, polyethylene oxide, pyrolytic carbon, polyvinylpyridine copolymers, polyamide polymers/copolymers polyimide polymers/copolymers, ethylene vinylacetate copolymer and/or polyether sulfones. The primer layer can have any suitable thickness, including between about 0.01 μm and 5.00 μm.

Medical Devices

The coatings may be applied to implantable or insertable medical devices of various configurations and functions. Typical subjects (also referred to herein as “patients”) are vertebrate subjects (i.e., members of the subphylum cordata), including, mammals such as cattle, sheep, pigs, goats, horses, dogs, cats and humans. Typical sites for placement of the medical devices include the coronary and peripheral vasculature (collectively referred to herein as the vasculature), heart, esophagus, trachea, colon, gastrointestinal tract, biliary tract, urinary tract, bladder, prostate, brain and surgical sites. Where the medical device is inserted into the vasculature, for example, the therapeutic agent may be released to a blood vessel wall adjacent the device, and may also be released to downstream vascular tissue as well.

The medical device of the invention may be any device that is introduced temporarily or permanently into the body for the prophylaxis or therapy of a medical condition. For example, such medical devices may include, but are not limited to, stents, stent grafts, vascular grafts, catheters, guide wires, balloons, filters (e.g. vena cava filters), cerebral aneurysm filler coils, intraluminal paving systems, sutures, staples, anastomosis devices, vertebral disks, bone pins, suture anchors, hemostatic barriers, clamps, screws, plates, clips, slings, vascular implants, tissue adhesives and sealants, tissue scaffolds, myocardial plugs, pacemaker leads, valves (e.g. venous valves), abdominal aortic aneurysm (AAA) grafts, embolic coils, various types of dressings, bone substitutes, intraluminal devices, vascular supports, or other known bio-compatible devices.

In general, intraluminal stents for use in connection with the present invention typically comprise a plurality of apertures or open spaces between metallic filaments (including fibers and wires), segments or regions. Typical structures include: an open-mesh network comprising one or more knitted, woven or braided metallic filaments; an interconnected network of articulable segments; a coiled or helical structure comprising one or more metallic filaments; and, a patterned tubular metallic sheet (e.g., a laser cut tube). Examples of intraluminal stents include endovascular, biliary, tracheal, gastrointestinal, urethral, ureteral, esophageal and coronary vascular stents. The intraluminal stents of the present invention may be, for example, balloon-expandable or self-expandable. Thus, although certain embodiments of the present invention will be described herein with reference to vascular stents, the present invention is applicable to other medical devices, including other types of stents.

In one embodiment of the present invention, the medical device comprises an intraluminal stent. The stent may be self-expanding or balloon-expandable and may be a bifurcated stent, a stent configured for any blood vessel including a coronary arteries and peripheral arteries (e.g., renal, Superficial Femoral, Carotid, and the like), a urethral stent, a biliary stent, a tracheal stent, a gastrointestinal stent, or an esophageal stent.

The stent or other medical device of the invention may be made of one or more suitable biocompatible materials such as stainless steel, nitinol, MP35N, gold, tantalum, platinum or platinum irdium, niobium, tungsten, iconel, ceramic, nickel, titanium, stainless steel/titanium composite, cobalt, chromium, cobalt/chromium alloys, magnesium, aluminum, or other biocompatible metals and/or composites or alloys such as carbon or carbon fiber.

Methods of Treatment

A method of treatment according to the present invention may include inserting into a patient a coated medical device having any of the configurations described above. For example, when the medical device is a stent coated by the coating methods described above, the method of treatment involves implanting the stent into the vascular system of a patient and allowing the therapeutic agent(s) to be released from the stent in a controlled manner, as shown by the drug elution profile of the coated stent.

In one preferred embodiment, the coated medical devices are implanted to treat peripheral vascular disease, for example by implanting the coated medical device in a peripheral artery. Peripheral vascular disease (PVD) is a common condition with variable morbidity affecting mostly men and women older than 50 years. Peripheral vascular disease of the lower extremities comprise a clinical spectrum that goes from asymptomatic patients, to patients with chronic critical limb ischemia (CLI) that might result in amputation and limb loss. Critical limb ischemia is a persistent and relentless problem that severely impairs the patient functional status and quality of life, and is associated with an increased cardiovascular mortality and morbidity. It can present acutely (i.e. distal embolization, external compression, acute thrombosis, etc.) or, in the majority of cases, as chronic CLI. Based on incidence rates extrapolated to today's increasingly aging population, PVD affects as many as 10 million people in the United States (Becker GJ, McClenny TE, Kovacs ME, et al., “The importance of increasing public and physician awareness of peripheral arterial disease,” J Vasc Interv Radiol., 13(1):7-11 (January 2002)). As the population ages, the family physician will be faced with increasing numbers of patients complaining of symptoms of lower extremity PVD. Nearly one in four of the approximately 60,000 people screened annually through Legs for Life, a nationwide screening program, are determined to be at moderate to high risk of lower extremity PVD and are referred to their primary care physicians for diagnosis (data collected by the Society of Cardiovascular and Interventional Radiology) (Becker G J, McClenny T E, Kovacs M E, et al., “The importance of increasing public and physician awareness of peripheral arterial disease,” J Vasc lnterv Radiol., 13(1):7-11 (January 2002)).

Chronic critical limb ischemia is defined not only by the clinical presentation but also by an objective measurement of impaired blood flow. Criteria for diagnosis include either one of the following (1) more than two weeks of recurrent foot pain at rest that requires regular use of analgesics and is associated with an ankle systolic pressure of 50 mm Hg or less, or a toe systolic pressure of 30 mm Hg or less, or (2) a nonhealing wound or gangrene of the foot or toes, with similar hemodynamic measurements. The hemodynamic parameters may be less reliable in patients with diabetes because arterial wall calcification can impair compression by a blood pressure cuff and produce systolic pressure measurements that are greater than the actual levels. Ischemic rest pain is classically described as a burning pain in the ball of the foot and toes that is worse at night when the patient is in bed. The pain is exacerbated by the recumbent position because of the loss of gravity-assisted flow to the foot. Ischemic rest pain is located in the foot, where tissue is farthest from the heart and distal to the arterial occlusions. Patients with ischemic rest pain often need to dangle their legs over the side of the bed or sleep in a recliner to regain gravity-augmented blood flow and relieve the pain. Patients who keep their legs in a dependent position for comfort often present with considerable edema of the feet and ankles. Nonhealing wounds are usually found in areas of foot trauma caused by improperly fitting shoes or an injury. A wound is generally considered to be nonhealing if it fails to respond to a four- to 12-week trial of conservative therapy such as regular dressing changes, avoidance of trauma, treatment of infection and debridement of necrotic tissue. Gangrene is usually found on the toes. It develops when the blood supply is so low that spontaneous necrosis occurs in the most poorly perfused tissues.

Treatment and prognosis of peripheral vascular disease can be influenced by lesion and patient characteristics, such as the site of the lesion, type of lesion (stenosis or occlusion, lesion length), arterial runoff, and clinical manifestation (Dormandy JA, Rutherford RB. Management of peripheral arterial disease (PAD): TASC Working Group. J Vasc Surg 2000; 31 (1 pt 2):S103-S106). Estimates of the 5-year patency rate of balloon dilation for femoropopliteal arterial disease range from as low as 12% in patients with an occlusion and critical ischemia to 68% in patients with a stenosis and claudication (Hunink MGM, Wong J B, Donaldson M C, Meyerovitz M F, Harrington D P. Patency results of percutaneous and surgical revascularization for femoropopliteal arterial disease. Med Decis Making 1994; 14:71-81). Bypass surgery for femoropopliteal arterial disease has been associated not only with higher long-term patency rates but also with a higher procedural morbidity, mortality, and a longer hospital stay (Hunink MGM, Wong J B, Donaldson M C, Meyerovitz M F, de Vries J A, Harrington D P. Revascularization for femoropopliteal disease. A decision and cost-effectiveness analysis. JAMA 1995; 274:165-171).

Methods of treating peripheral vascular disease, including critical limb ischemia, preferably comprise the endovascular implantation of one or more coated medical devices provided herein. Atherosclerosis underlies many cases of peripheral vascular disease, as narrowed vessels that cannot supply sufficient blood flow to exercising leg muscles may cause claudication, which is brought on by exercise and relieved by rest. As vessel narrowing increases, critical limb ischemia (CLI) can develop when the blood flow does not meet the metabolic demands of tissue at rest. While critical limb ischemia may be due to an acute condition such as an embolus or thrombosis, most cases are the progressive result of a chronic condition, most commonly atherosclerosis. The development of chronic critical limb ischemia usually requires multiple sites of arterial obstruction that severely reduce blood flow to the tissues. Critical tissue ischemia can be manifested clinically as rest pain, nonhealing wounds (because of the increased metabolic requirements of wound healing) or tissue necrosis (gangrene).

The coated medical device can be implanted in any suitable body vessel. The configuration of the implantable frame can be selected based on the desired site of implantation. For example, for implantation in the superficial artery, popliteal artery or tibial artery, frame designs with increased resistance to crush may be desired. For implantation in the renal or iliac arteries, frame designs with suitable levels of radial force and flexibility may be desired. Preferably, a coated vascular stent is implanted in a non-coronary peripheral artery, such as the iliac or renal arteries.

In one embodiment, a medical device comprising a balloon-expandable frame portion coated with a therapeutic agent covered by a layer of biodegradable elastomer polymer can be endoluminally delivered to a point of treatment within an infrapopliteal artery, such as the tibial or peroneal artery or in the iliac artery, to treat CLI. For treating focal disease conditions, coated balloon-expandable medical devices can comprise an expandable frame attached to a coating. The frame can be also be formed from a bioabsorbable material, or comprise a coating of bioabsorbable material over at least a portion of the frame. The frame can be configured to include a barb or other means of securing the medical device to the wall of a body vessel upon implantation.

In another embodiment, a coated medical device can be a self-expanding device such as a coated NITINOL stent configured to provide a desirable amount of outward radial force to secure the medical device within the body vessel. The medical device can be preferably implanted within the tibial arteries for treatment of CLI. For instance, the coated medical device can be configured as a vascular stent having a self-expanding support frame formed from a superelastic self-expanding nickel-titanium alloy coated with a metallic bioabsorbable material and attached to a graft material. A self-expanding frame can be used when the body vessel to be stented extends into the distal popliteal segment. The selection of the type of implantable frame can also be informed by the possibility of external compression of an implant site within a body vessel during flexion of the leg.

Methods for delivering a medical device as described herein to any suitable body vessel are also provided, such as a vein, artery, biliary duct, ureteral vessel, body passage or portion of the alimentary canal.

Although exemplary embodiments of the invention have been described with respect to the treatment of complications such as restenosis following an angioplasty procedure, the local delivery of therapeutic agents may be used to treat a wide variety of conditions using any number of medical devices. For example, other medical devices that often fail due to tissue ingrowth or accumulation of proteinaceous material in, on, or around the device may also benefit from the present invention. Such devices may include, but are not limited to, intraocular lenses, shunts for hydrocephalus, dialysis grafts, colostomy bag attachment devices, ear drainage tubes, leads for pace makers, and implantable defibrillators.

A consensus document has been assembled by clinical, academic, and industrial investigators engaged in preclinical interventional device evaluation to set forth standards for evaluating drug-eluting stents such as those contemplated by the present invention. See “Drug-Eluting Stents in Preclinical Studies—Recommended Evaluation From a Consensus Group” by Schwartz and Edelman (available at “http://www.circulationaha.org”) (incorporated herein by reference).

Methods for delivering a medical device as described herein to any suitable body vessel are also provided, such as a vein, artery, biliary duct, ureteral vessel, body passage or portion of the alimentary canal.

While many preferred embodiments discussed herein discuss implantation of a medical device in a vein, other embodiments provide for implantation within other body vessels. In another matter of terminology there are many types of body canals, blood vessels, ducts, tubes and other body passages, and the term “vessel” is meant to include all such passages.

The invention includes other embodiments within the scope of the claims, and variations of all embodiments, and is limited only by the claims made by the Applicants.

### EXAMPLES

### Example 1

### Stents Coated with Single Layer of Therapeutic Agent

Paclitaxel was applied to Zilver® stents (nitinol stents manufactured by Cook Inc., Bloomington, Ind.) ranging in size from 6×20 mm to 14×80 mm, as follows. First, paclitaxel was dissolved in ethanol to form a 2.4 mM solution. The paclitaxel was substantially dissolved within about 30 minutes, using sonication. The paclitaxel solution was then filtered through a 0.2 micron nylon filter and collected in a flask. Approximately 10 ml of ethanol was filtered through a 0.2 micron nylon filtered and then transferred into a reservoir connected to a spray gun nozzle. This solution was then used to set the flow rate of the spray gun to the target flow rate of approximately 5.7 ml/min. Stents were mounted on a mandrel assembly positioned in the lumen of the stent, including a silicon tube covering a steel rod. This assembly masked the lumens of the stents and substantially prevented the lumens from being coated.

Approximately 25 ml of the filtered paclitaxel solution was added to the spray gun reservoir, and the solution was sprayed onto the stents using a conventional pressure spray gun manufactured by Badger (Model No. 200), in a HEPA filtered hood, with a fluid dispensing system connected to a pressure source (nitrogen) until the target dose of paclitaxel was reached. Adjustments on the system were used to control the spray pattern and the amount of fluid dispensed. The spray gun was aligned with the stents by setting a laser beam even with the nozzle of the spray gun and positioning the stents so that the laser beam was located at approximately ¼ the distance from the top of the stents. The spray gun, which was positioned parallel to the hood floor and at a horizontal distance of approximately 5-7 inches from the stents, was then passed over the surface of the stents until a predetermined volume of spray was dispensed. The stents were then rotated approximately 90° and the spraying procedure was repeated until the entire circumference of each stent was coated. The movement of the gun was slow enough to allow the solvent to evaporate before the next pass of the gun. Each spray application covered approximately 90° of the circumference of the stents. The stents were kept at ambient temperature and humidity during the spraying process. After substantially all of the solvent had evaporated, a coating of paclitaxel was left on the stent.

### Example 2

### Single Layer of PLA Over Single Layer of Paclitaxel on a Stent Using Pressure Gun Spray Coating Method

Paclitaxel was applied to Zilver® stents (nitinol stents manufactured by Cook Inc., Bloomington, Ind.) ranging in size from 6×20 mm to 14×80 mm, as follows. First, a layer of paclitaxel was applied as described in Example 1.

After the paclitaxel layer air dried, a layer PLA was then spray deposited over the paclitaxel coating using the same type of pressure spray coating apparatus as Example 1. A solution of approximately 2-4 g/L of PLA in dichloromethane was prepared, filtered over a 0.2 micron nylon filter, and collected in a flask. The solution was then sprayed over the coating of paclitaxel using a procedure similar to the one described above with respect to paclitaxel. For PLA, however, the spraying is performed at two different heights. First, the stents were positioned approximately 115 mm from the hood floor, sprayed, and rotated until the circumference of the top portion of the stents was coated. Next, the stents were positioned approximately 130 mm from the hood floor, sprayed, and rotated until the circumference of the bottom portion of the stents was coated.

Three different stent systems were tested, as described above with respect to FIG. 5B. Specifically, the elution profile 610 was obtained from a vascular stent having first layer of 69 μg paclitaxel deposited on the abluminal surface of the stent, and 173 μg of poly(D,L)Iactic acid deposited in a second layer over the paclitaxel. A second elution profile 620 was obtained from a two-layer coating having a first layer of 5 μg paclitaxel deposited on the abluminal surface of the stent, and 73 μg of poly(D,L)lactic acid deposited in a second layer over the paclitaxel. A third elution profile 550 was obtained from a two-layer coating having a first layer of 69 μg paclitaxel deposited on the abluminal surface of the stent, and 88 μg of poly(D,L)lactic acid deposited in a second layer over the paclitaxel. Numerical data for some of the resulting coated stents (obtained using a UV detection of paclitaxel in the modified porcine serum elution assay described Example 5) are shown below in Table 6.

TABLE 6

| | % PTX Dissolved | | |
|---|---|---|---|
| | 69 μg | 5 μg | 69 μg |
| Time | PTX/173 μg | PTX/73 μg | PTX/88 μg |
| (hrs) | PLA | PLA | PLA |
| 0 | 0.00 | 0.00 | 0.00 |
| 6 | 39 | 28 | 56 |
| 12 | 48 | 33 | 65 |
| 24 | 53 | 37 | 74 |
| 30 | 56 | 40 | 76 |
| 46 | 59 | 44 | 79 |
| 68 | 61 | 49 | 82 |
| 90 | 62 | 53 | 84 |
| 110 | 63 | 54 | 85 |
| 113 | 63 | 54 | 86 |
| 132 | 64 | 56 | 87 |
| 154 | 65 | 58 | 87 |
| 175 | 66 | 59 | 88 |
| 176 | 66 | 59 | 88 |
| 197 | 67 | 61 | 88 |
| 221 | 68 | 63 | 89 |
| 243 | 69 | 64 | 89 |
| 289 | 70 | 66 | 89 |
| 329 | 70 | 67 | 89 |
| 375 | 71 | 68 | 89 |
| 393 | 71 | 69 | 89 |
| 415 | 71 | N/A | 90 |
| 461 | 72 | 70 | 90 |
| 480 | 72 | 70 | 90 |
| 507 | 72 | 71 | 90 |

### Example 3

### Single Layer of PLA Over Single Layer of Paclitaxel on a Stent Using Electrostatic Spray Deposition Method

Approximately 1-25 micrograms of paclitaxel was applied to a Zilver® stent by dissolving the paclitaxel in ethanol (using sonication) at a concentration of about 2.4 mM and applying the solution to a stent with an electrostatic spraying apparatus (Teronics Development Corp.). Specifically, the solution was loaded into a 20 mL syringe, which was then mounted onto a syringe pump and connected to a tub that carries the solution to a spray head. The syringe pump was then used to purge the air from the solution line and prime the line and spray nozzle with solution. An electrical connection to the nozzle supplied the required voltage. The stent was then slipped over a mandrel (Teronics Development Corp., 6 mm×60 mm) until one end of the stent made contact with the electrical connection at one end of the mandrel. This connection provided the ground potential to the stent. The motor was then activated and the stent was rotated at a constant, slow speed. The syringe pump was then activated to supply the nozzle with a consistent flow of solution, and the power supply was activated to provide a charge to the solution and cause the solution to jump the air gap and land on the stent surface. As the coated surfaces were rotated away from the spray path, the volatile portion of the solution evaporated leaving a coating of therapeutic agent behind. The stent continued to rotate in the spray pattern until the desired dose had accumulated.

During the coating process, the stent was kept at ambient temperature and humidity, the solution was pumped at a rate of about 0.5-10 mL/hr, preferably about 0.5-8 mL/hr through the spray gun (which was placed at a horizontal distance of approximately 6 cm from the stents), and the bias voltage between the spray nozzle and the stent was approximately 5-20 kilovolts, preferably about 12 kilovolts. Substantially all of the solvent had evaporated during the spraying process, leaving a dose of about 0.1 μg-3 μg of paclitaxel per mm² on the abluminal surface area of the stent.

PLA was then applied over the paclitaxel coating by dissolving approximately 1.2 g/L (+/−0.3 g/L) of PLA in a 2:1 v/v mixture of methanol and dichloromethane to obtain a finer spray that is more conducive to electrostatic spraying than the spray created by dissolving paclitaxel in dichloromethane alone. The solution was then applied to the stent by using an electrostatic spray deposition process as described above.

### Example 4

### Single Layer of PLA Over Single Layer of Paclitaxel on a Stent Using Ultrasonic Deposition Method

Paclitaxel was applied to a Zilver® stent by dissolving Paclitaxel in ethanol at a concentration of about 2.4 mM. The therapeutic agent is applied to a stent with the Pressure Gun or Ultrasonic Nozzle.

Once the stents are coated with the Paclitaxel, PLA is applied by dissolving 2 to 4 g/L in dichloromethane. The solution is then applied by using the ultrasonic nozzle. The solution is loaded into a 10.0 mL syringe, which is mounted onto a syringe pump and connected to a tube that carries the solution to a spray head. The syringe pump was then used to purge the air from the solution line and prime the line and spray nozzle with the solution. The ultrasonic nozzle is arranged such that excitation of the piezoelectric crystals generates a transverse standing wave along the length of the nozzle. So the solution introduced onto the atomizing surface absorbs some of the vibrational energy, setting up wave motion in the liquid. For the liquid to atomize, the vibrational amplitude of the atomizing surface must be carefully controlled. The coating chamber is purged with nitrogen to displace any oxygen in the system. The coating method is created and the system is set-up using the corresponding parameters. After that, one end of the stent is slipped onto a mandrel and half of the stent is coated. The nozzle is manually aligned to the tip of the stent and the middle of the stent. These position numbers are used for the coating program when the syringe pump is actually activated. The stent is turned over and the other half is coated. During the process, the stent is kept at ambient temperature and in a closed chamber.

TABLE 7
Process Parameters for Ultrasonic Coating

| | Coating | Rotation | Nozzle | | |
|---|---|---|---|---|---|
| Flow rate | velocity | Speed | Power | Process | Distance |
| (mL/min) | (in/sec) | (rpm) | (watts) | Gas (psi) | (mm) |
| 0.01-2 | 0.005-0.5 | 30-150 | 0.9-1.2 | 0.1-2.5 | 1-25 |

### Example 5

### Porcine Serum Assay to Measure Paclicaxel Elution from a Coated Vascular Stent

The porcine serum (1500 mL) was thawed in a water bath at 37° C. Once the porcine serum was thawed, heparin was added to avoid coagulation. 0.104 mL of a 6 g/L Heparin solution in water is added per mL of porcine serum. The pH of the media is regulated using an aqueous solution of acetic acid (20% v/v). The acidic solution is added to the porcine serum until the pH meter indicates a pH of 5.6±0.3. The initial and final temperature and the initial and final pH are recorded. Once the porcine serum is ready, 7-250 mL Erlenmeyer flasks are filled with 202.00±0.05 g. A stir bar should be placed in each flask and the lids are placed on the corresponding Erlenmeyer flask. The flask corresponding to the violet chamber, which is the control channel, is spiked with 10 μL of an ethanolic 1.2 mM PTX solution.

The 250 mL Erlenmeyer flasks are placed on the 10-well stir plate and it is ensured that the solutions are being stirred. The inlet and outlet tubes are placed into appropriate places in the flask. The stents are placed in the corresponding channel. The cells are assembled. After setting the time points, the cells are inserted and the test is started and allowed to run for the established period of time. A 4 L beaker with DiW and a lint free cloth is placed into the water to clean the auto-sampler head after the sample is collected. 4-mL samples are collected and sent to a UV-VIS spectrophotometer (or other suitable detector) to detect the presence of the therapeutic agent (e.g., paclitaxel absorption at 227 nm), or transferred to a cryovial tube and placed in the freezer at −25° C., and then shipped on dry ice for later analysis.

### Example 6

### Measurement of % Elution of Paclitaxel from Paclitaxel:PLA Coated Vascular Stents After 20 Days in Porcine Serum

Six ZILVER® stents were coated with a two-layer coating as described in Example 2, except that the amount of paclitaxel and poly(D,L)lactic acid (PLA) was varied in each stent as indicated below. The two layer coating consisted of a first layer of paclitaxel covered with a second layer of PLA on the abluminal surface only. The percentage of paclitaxel eluted from the stent after 20 days of the porcine serum assay of Example 5 was measured and recorded in Table 8 below.

TABLE 8

| | 75 +/− 112 μg | 134 +/− 10 μg | 170 +/− 26 μg |
|---|---|---|---|
| | PLA | PLA | PLA |
| PTX/PLA | (0.94 μg/mm²) | (1.6 μg/mm²) | (2.0 μg/mm²) |
| 5.3 +/− 0.3 μg | 70% | | 42% |
| PTX | | | |
| (0.06 μg/mm²) | | | |
| 7.8 +/− 0.4 μg | | 54% | |
| PTX | | | |
| (0.1 μg/mm²) | | | |
| 23 +/− 2.5 μg PTX | | 48% | |
| (0.3 μg/mm²) | | | |
| 69 +/− 4.2 μg PTX | 90% | | 72% |
| (1.0 μg/mm²) | | | |

### Example 7

### Durability of Coated Stents

The durability of vascular stents coated in Examples 2-4 were evaluated by measuring weight loss from sterilization, as indicated in Table 9 below. More durable coatings were obtained by using lower concentrations of paclitaxel in ethanol (e.g., preferably about 1.2 or 0.7 mM paclitaxel in ethanol) to apply the coating layer of paclitaxel.

TABLE 9
Durability of Coated Stents

| | Weight Before | Weight After | |
|---|---|---|---|
| Stent | Sterilization (μg) | Sterilization (μg) | Δ (μg) |
| Pressure Gun | 91800 | 91801 | +1 |
| Electrostatic | 90726 | 90720 | −6 |
| Ultrasonic | 91927 | 91928 | +1 |

The durability of additional vascular stents coated PLA of different molecular weights over paclitaxel was also investigated. Three sets of 10 6×20 mm stents were each coated with a 2.4 mM PTX solution in EtOH, achieving an average dose of 74 μg. Then, the coated stents were over coated with different molecular weights of PLA. The molecular weights of the PLA were: 75,000; 240,000 and higher than 240,000 Dalton. There were two doses: a 1:1 and 1:2 (PTX:PLA) ratio. Table 10 shows the nominal doses of PLA.

TABLE 10
Nominal Doses of PLA Coating

| | PLA | Amount PLA |
|---|---|---|
| Stent # | Used | (mg) |
| 1 | 75000 | 152 |
| 2 | | 154 |
| 3 | | 70 |
| 4 | | 150 |
| 5 | | 74 |
| 6 | | 72 |
| 7 | | 80 |
| 8 | | 146 |
| 9 | 240000 | 105 |
| 10 | | 80 |
| 11 | | 194 |
| 12 | | 130 |
| 13 | | 151 |
| 14 | | 143 |
| 15 | | 81 |
| 16 | | 70 |
| 17 | >240000 | 131 |
| 18 | | 114 |
| 19 | | 126 |
| 20 | | 99 |
| 21 | | 97 |
| 22 | | 95 |
| 26 | | 95 |
| 27 | | 86 |

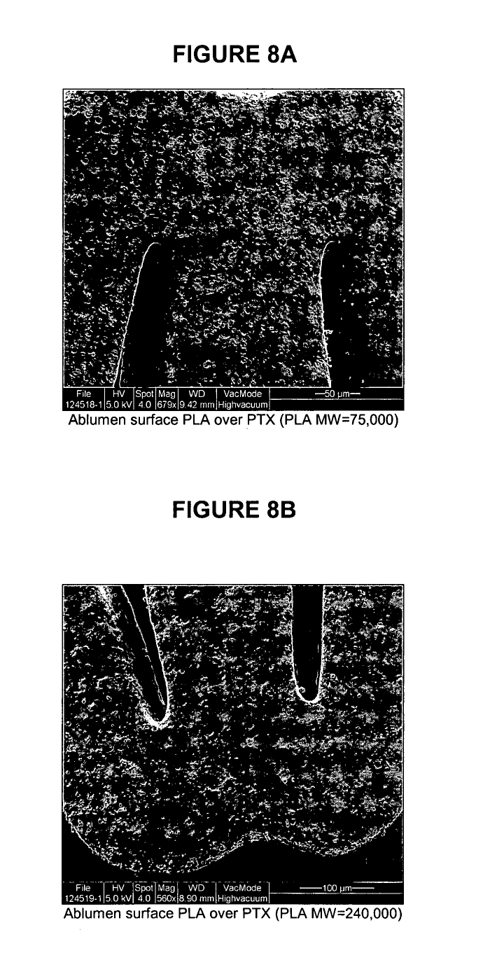

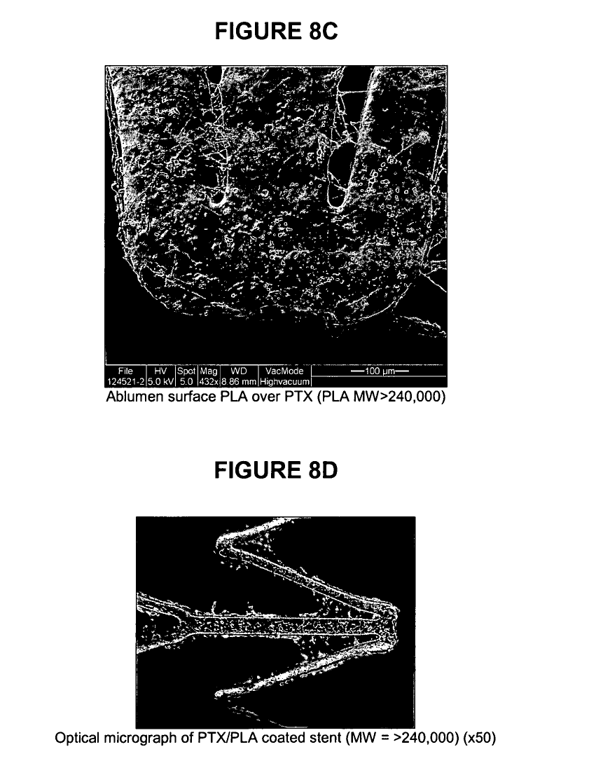

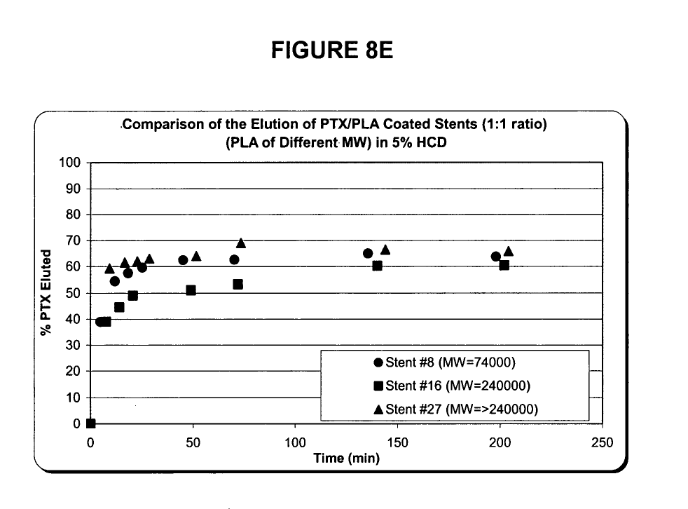

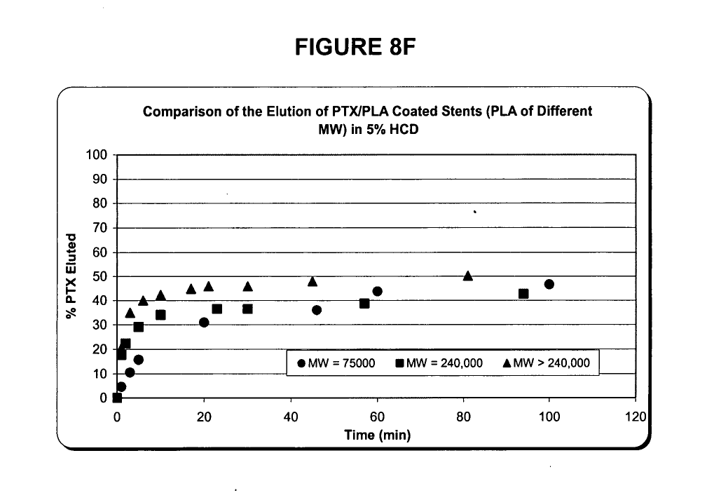

SEM images were taken in order to compare the smoothness and uniformity of the coatings. FIG. 8A shows a representative SEM micrograph of the surface of the PLA coating over paclitaxel, with a PLA molecular weight of 75,000 Da. The coating is uniform and there are not scratches or peeling. There are a lot of bead-like structures on the coating. However, after sterilization, the beads typically disappear. FIG. 8B shows a SEM micrograph of a PLA coating over the paclitaxel layer on the stent using the 240,000 Da PLA. FIG. 8C shows the webbing formed when coating with a PLA molecular weight higher than 240,000 Da. Furthermore, the >240,000 Da coating does not look as good as the other two. The coating has perforations and peeling in some areas.

To determine the durability of the stent, each coated stent was crimped to 5.5 French and loaded into a delivery system. After the process was completed, the coated stents were deployed in air. The stents were weighed showing that the stent lost less than 1% of its weight. After photographing and taking SEM of the stents, it was determined that the durability of the first two coatings are comparable, on the other hand the third one is not as good. The 75,000 Da PLA coating produced a very durable coating similar to the coating in FIG. 7C. The coating using the 240,000 Da was comparatively durable. However, there are areas where the coating was observed to be peeling off. The durability of the coating comprising the PLA with the molecular weight of greater than 240,000 is compromised by the webbing between the struts. An optical micrograph of this coating is shown in FIG. 8D, with webbing and the coating clearly damaged after the coated stent was crimped.

Elution profiles were obtained for six of the coated stents in 5% HCD in order to determine the differences in elution between the different PLAs. Surprisingly, FIG. 8E shows that the elution of the stent coated with a PLA with MW higher than 240,000 is somewhat faster than the other two, lower molecular weight PLA coatings. The high elution rate of the highest molecular weight PLA may be due to cracking in the coating, allowing the paclitaxel to elute more rapidly through the cracks in the PLA. Notably, the durability of the highest molecular weight PLA coating is significantly compromised, as seen in the roughness and the overall cracked appearance of the surface of the coating. FIG. 8E shows the elution profile for the PTX/PLA (1:1 ratio) coated stents using PLA of different MW in 5% HCD. FIG. 8F shows the elution profile for the PTX/PLA (1:2 ratio) coated stents using PLA of different MW in 5% HCD. Notably, the PLA with a >240,000 kDa molecular weight is also the fastest eluting coating in FIG. 8F.

### Example 8

### Interferometry of Coating Surface

Using an interferometer, the surface roughness of a PLA coating applied in Example 4 was evaluated in two regions of the coating each measuring about 0.1 mm×0.1 mm. The mean roughness in the first region was about 288 nm, with a peak/valley distabce of about 615 nm. The mean roughness in the second region was about 75 nm, with a peak/valley distance of about 1420 nm. These measurements indicate a surface roughness on the sub-micron level.

### Comparative Example 9

### Single Layer of Paclitaxel/PLA Mixture

To determine the concentration of paclitaxel that would result in the most uniform and smooth coating of a therapeutic agent/polymer bioabsorbable polymer mixture, experiments were performed with different concentrations of paclitaxel at a fixed weight ratio of paclitaxel to PLA of 1:1 up to about 1:5. As shown in Table 5, the results indicate that the most uniform and smooth coating was obtained by using low concentrations of paclitaxel, such as a 2.4 mM solution of paclitaxel in ethanol or methanol.

TABLE 11

| Stent | Concen- | | |
|---|---|---|---|
| size | tration of | Amount of | |
| (mm) | PTX (mM) | PTX (μg) | Observations |
| 6 × 20 | 3.12 | 59.9 | Coating not uniform; a lot of |
| | | | webbing present; stent looked white |
| 7 × 40 | 1.73 | 55.3 | Uniform coating; smooth and |
| | | | slightly textured |
| 6 × 20 | 0.624 | Not avail. | Uniform coating; thin and smooth |

### Example 8

### Animal Testing

An animal implant study was performed using 6×20 mm Zilver® stents coated with 0, 0.06, 0.3, and 0.9 μg/mm² of paclitaxel (PTX), nominal doses are 0, 5, 24, and 72 μg respectively, along with a top-coat of either 60 or 180 μg poly(D,L-lactide) (PLA). The stents were implanted in normal domestic porcine iliac and femoral arteries. The PTX levels on the stents were evaluated after 15±2 days of implantation. The evaluation involved an extraction step followed by high performance liquid chromatography (HPLC). It was predicted based on porcine serum elution assays (Example 5) that the amount of PTX remaining on the stent will be between 10-40% of the total dose.

The PTX remaining on each stent was extracted in 100% ethanol (EtOH). The PTX in the ethanolic solution will be quantified using HPLC per validated BAS method SAP #820-0564 The Measurement of Paclitaxel on Stainless Steel Stents. After the pigs were euthanized, the stented arteries were excised. Isolation of the stent was done by carefully opening the artery longitudinally and separating it from the stent. Any remaining tissue on the isolated stent was removed with tweezers. Each stent was placed in an individually labeled vial. The vials identified with the pig number, stent number, and nominal dose of PTX on the stent followed by an “A” or “B” (“A” for stent extraction and “B” for tissue rinse). Approximately 5 mL of EtOH was measured accurately (by weight) in trace clean vials. The stents were added to their respective vials and vortexed for approximately 10 seconds, placed on the shaker for 15 minutes, and vortexed a second time for 10 seconds. The stents were removed from the vials and EtOH samples were centrifuged for 5 minutes at approximately 2500 rpm. In addition, to account for any PTX that may have been peeled off when removing the stent from the artery, the tissue was rinsed with about 3-5 mL of EtOH measured accurately (the volume of EtOH will be measured gravimetrically by collecting the tissue rinse in a tared trace clean vial and then reweighing after all the rinse has been collected). The ethanolic solution was centrifuged for 5 minutes at approximately 2500 rpm.

The paclitaxel remaining on the explanted coated stents was analyzed as described in Example 5, and the amount of paclitaxel (PTX) remaining on the stent is shown in Table 9 below. These results indicate that about 80% (stent 2) to about 54% (stent 6) of the paclitaxel eluted from the stents within the blood vessel after about 15 days (about 360 hrs). Comparing the results from the animal studies with the results from the porcine serum assay results in FIGS. 5A-5C indicates that the modified porcine serum elution medium described in Example 5 can be used to predict in vivo rates of therapeutic agent release.

TABLE 12
Animal Testing: PTX/PLA Coated Zilver ® 6 × 20 mm Stents

| | | PTX Doses | | PLA Doses | | | % PTX |
|---|---|---|---|---|---|---|---|
| Stent | Swine | Target | Average | Target | Actual | Actual | Remaining on |
| # | # | (μg) | (μg) | (μg) | (μg) | Location | Stent |
| 5 | 486 | 0 | 0 | 180 | 105 | Left Iliac | 1.43 |
| 18 | | 5 | 7 | 60 | 46 | Right Iliac | 22.53 |
| 21 | 487 | 0 | 0 | 180 | 103 | Celiac | 0.00 |
| 2 | | 5 | 7 | 60 | 45 | Superior | 20.52 |
| | | | | | | Mesenteric | |
| 13 | 488 | 0 | 0 | 180 | 108 | Left Iliac | 0.00 |
| 6 | | 5 | 7 | 60 | 48 | Right Iliac | 45.89 |
| 7 | 486 | 24 | 23 | 60 | 40 | Left Femoral | 5.35 |
| 44 | | 72 | 69 | 180 | 115 | Right Femoral | 15.80 |
| 23 | 487 | 24 | 23 | 60 | 47 | Left Femoral | 6.60 |
| 16 | | 72 | 69 | 180 | 125 | Right Femoral | 26.08 |
| 3 | 488 | 24 | 23 | 60 | 50 | Left Femoral | 13.29 |
| 20 | | 72 | 69 | 180 | 122 | Right Femoral | 13.58 |

### Example 9

### Animal Testing 15-Day Explant Follow-up

An animal implant study was performed according to the metod of Example 8 using 6×20 mm Zilver® stents coated with 0, 0.06, 0.3, and 0.9 μg/mm² of paclitaxel, nominal doses are 0, 5, 24, and 72 μg respectively, along with a top-coat of either 60 or 180 μg poly(D,L-lactide) (PLA). The stents were implanted in normal domestic porcine iliac and femoral arteries. The dose levels and durability data for each explanted stent (measured prior to implantation) is included in Tables 13 and 14. The data in Table 14 was obtained prior to implantation but after the coated stents were were crimped to about 5.5 F, loaded on a cathether delivery device and then deployed in air and subsequently re-weighed.

TABLE 13
Original doses of PTX & PLA: for explanted coated stents

| Data | PTX* (ug) | | | PLA (ug) | |
|---|---|---|---|---|---|
| Target Dose | 5 | 24 | 72 | 60 | 180 |
| Average Dose Achieved | 7 | 23 | 69 | 51 | 114 |
| Standard Deviation | 0.80 | 3.03 | 5.94 | 4.67 | 22.94 |

TABLE 14
Durability: Δ Weight

| | Average | Average | |
|---|---|---|---|
| | Weight | Weight | |
| | After | After | |
| | Coating | Deployed | Δ |
| Stent | (μg) | (μg) | Weight |
| 1 | 92875 | 92879 | 4 |
| 2 | 92805 | 92809 | 4 |
| 3 | 90001 | 90011 | 9 |
| 4 | 91496 | 91506 | 9 |
| 5 | 92801 | 92810 | 8 |
| 6 | 93115 | 93115 | 1 |

The following coated 6×20 mm Zilver® were implanted:

| | |
|---|---|
| Polymer only | 180 μg (2.25 μg/mm²) PLA |
| Low dose PTX | 5 μg PTX (0.06 μg/mm²); 60 μg PLA |
| High dose PTX | 24 μg PTX (0.3 μg/mm²); 60 μg PLA |
| 3X PTX dose | 72 μg PTX (0.9 μg/mm²); 180 μg PLA |

Two coated sterilized stents (polymer only and 5 μg PTX) were implanted 20 mm apart in the Iliac artery and two other coated sterilized stents were implanted in the femoral artery (24 μg PTX and 72 μg PTX) The amount of paclitaxel remaining on the explanted stents after approximately 14 days after implantation was calculated by measuring an elution profile of each explanted stent using HPLC and an elution media. Table 15 shows the average percentage of paclitaxel delivered into the porcine arterial implant sites as a function of paclitaxel stent loading:

TABLE 15
Measurement of Paclitaxel on Stents Explanted at 15 days

| PTX Dose (μg) | Average Delivered PTX (%) |
|---|---|
| 5 | 70 |
| 23 | 92 |
| 69 | 82 |

### Example 10

### Animal Testing 30-Day Angiogram Follow-up

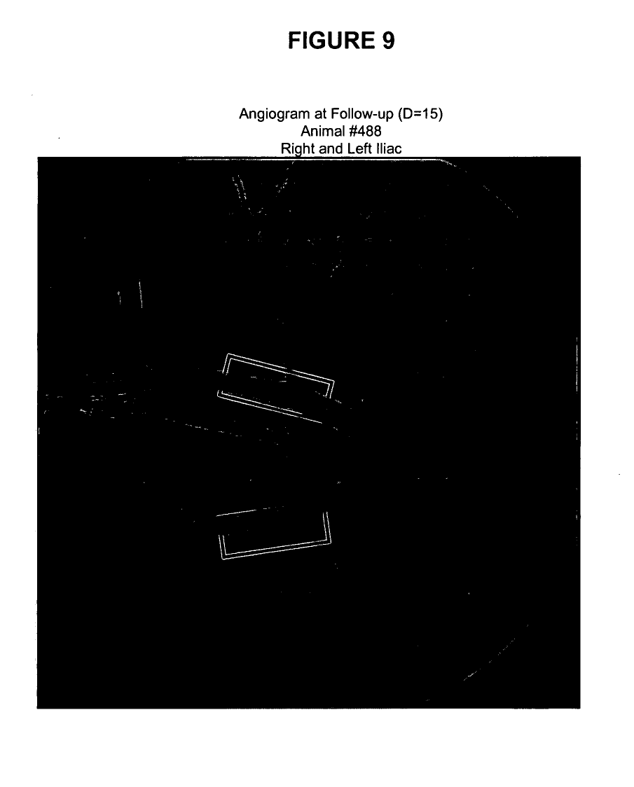

After the animal implant study was performed according to the metod of Example 8, an angiogram was performed to view the right and left iliac vascular sites of coated stent implantation, prior to explant of the stents. A representative angiogram is included as FIG. 9, showing minimal stenosis (less than about 5%) and no edge effect. The rectangular boxes indicate the site of coated stent implantation.

We claim:

1. A method of delivering a therapeutic agent to a peripheral blood vessel comprising the steps of:
a. providing a coated vascular stent comprising
i. a radially-expandable vascular stent having an abluminal side and a luminal side defining a substantially cylindrical lumen and being movable from a radially expanded configuration to a radially compressed configuration; and
ii. a multi-layer coating on the abluminal surface, the coating comprising two layers including
1. a first layer comprising between about 0.05 and 1.00 μg of a taxane therapeutic agent per mm² of the surface, and less than 0.1 μg of a polymer; the first layer positioned between the surface and a second layer; and
2. the second layer positioned over the first layer and comprising between about 0.05 and 20 mg of a biodegradable elastomer per mm² of the surface, the biodegradable elastomer having a molecular weight of 75,000-240,000 kDa, and being present in an amount between 1 and 20 times the weight of the therapeutic agent in the first layer;
b. intralumenally inserting the coated vascular stent into the blood vascular system using a means for intralumenal delivery comprising a catheter;
c. positioning the coated vascular stent within a peripheral artery; and
d. radially expanding the coated vascular stent within the peripheral artery so as to place the coated vascular stent in contact with a portion of a wall of the peripheral artery in a manner effective to deliver the therapeutic agent to the wall of the peripheral artery.

2. The method of claim 1, wherein the biodegradable elastomer comprises a polymer or copolymer including at least one polymer selected from the group consisting of: poly(lactic acid), poly(glycolic acid), poly(4-hydroxybutyrate) and poly(glycerol-sibacate).

3. The method of claim 2, wherein the biodegradable elastomer is a poly(lactic acid) selected from from the group consisting of: poly(L-lactic acid), poly(D-lactic acid) and poly(D,L-lactic acid).

4. The method of claim 1, wherein the coated vascular stent is placed within a peripheral artery selected from the group consisting of: an iliac artery and a femoral artery.

5. The method of claim 1, wherein the taxane therapeutic agent is paclitaxel, the coated vascular stent comprises about 0.06 to 0.90 μg of paclitaxel per mm² of the abluminal surface, and the luminal surface comprises less than 0.01 μg of paclitaxel.

6. The method of claim 5, wherein the biodegradable elastomer is poly(lactic acid), the second layer comprises less than 0.01 μg of paclitaxel and the first layer comprises less than 0.01 μg of the poly(lactic acid).

7. A method for coating an implantable medical device to form a drug delivery system, the method comprising the steps of:
a. providing an implantable medical device having a surface;
b. depositing a first layer consisting essentially of a hydrophobic therapeutic agent on the surface of the medical device by the steps of:
i. applying to the surface a first solution comprising a first solvent and a hydrophobic therapeutic agent dispersed in the first solvent, where the first solution does not contain a polymer;
ii. evaporating the first solvent to form the first coating layer consisting essentially of the therapeutic agent on the surface;
iii. repeating the application and evaporation steps until the first layer contains between about 0.05 and 1.00 μg of a hydrophobic therapeutic agent per mm² of the surface; and
c. depositing a second layer comprising a biodegradable elastomer over the first coating layer on the medical device to form a coated medical device by the steps of:
i. applying to the first layer a second solution comprising a second solvent and a biodegradable elastomer polymer dispersed in the second solvent, the biodegradable elastomer having a molecular weight of 75,000-240,000 kDa;
ii. evaporating the second solvent to form at least a portion of the second coating layer;
iii. repeating the application and evaporation steps until the weight of the biodegradable elastomer in the second layer is between 1 and 20 times greater than the weight of the therapeutic agent in the first layer.

8. The method of claim 7, wherein the first solution is a 0.5-5.0 mM solution of a taxane therapeutic agent.

9. The method of claim 7, wherein the first solution is a 0.5-2.5 mM solution of paclitaxel in an alcohol.

10. The method of claim 7, wherein the second solution has a concentration of 0.1-7.0 g of the biodegradable elastomer per L of the second solution.

11. The method of claim 7, wherein the second solution does not contain the therapeutic agent.

12. The method of claim 7, wherein the second solution consists of about 5.0 g of poly(lactic acid) per L of dichloromethane.

13. The method of claim 7 wherein the medical device is a radially-expandable vascular stent having an abluminal surface and a luminal surface defining a substantially cylindrical lumen and being movable from a radially expanded configuration to a radially compressed configuration, where the coating is deposited on the abluminal side of the vascular stent.

14. The method of claim 13, wherein the vascular stent has a radially expanded configuration having a diameter of about 2-10 mm, and a radially compressed configuration having a diameter of about 1.0-2.0 mm, and wherein the coating is deposited on the abluminal surface.

15. The method of claim 14, wherein the coating is deposited on the abluminal surface of vascular stent in the radially expanded configuration, and the method further comprises the steps of:
a. measuring the weight of the coating after depositing the second layer;
b. radially compressing the vascular stent from the radially expanded configuration to the radially compressed configuration; and
c. measuring a loss in coating weight of up to 5% of the coating weight after the coated vascular stent is compressed to the radially compressed configuration.

16. The method of claim 7 wherein the therapeutic agent is paclitaxel and the method further comprises the steps of:
a. contacting the coated medical device with a porcine serum elutable medium for 24 hours under a porcine serum elution assay; wherein the porcine serum elutable medium is prepared by adding 0.104 mL of a 6.0 g/L Heparin solution to porcine serum at 37° C. and adjusting the pH to 5.6+/−0.3 using a 20% v/v aqueous solution of acetic acid; and wherein the porcine serum elution assay is performed by contacting the implantable medical device with the porcine serum elutable medium at a flow rate of 16 mL/min; and
b. measuring an elution of paclitaxel from the first coating layer for 24 hours.

17. The method of claim 16, wherein the first coating layer contains less than 1.00 mg of paclitaxel per mm² of the abluminal surface area of the vascular stent and less than 40% of the paclitaxel elutes from the coated vascular stent after 24 hours of the porcine serum elution assay.

18. A coated implantable medical device comprising a coating configured to release a therapeutic agent adhered to a surface of the medical device, the coating comprising:
a. a first layer comprising between about 0.05 and 1.00 μg of a hydrophobic therapeutic agent per mm² of the surface, and less than 0.1 μg of a polymer; the first layer positioned between the surface and a second layer; and
b. the second layer positioned over the first layer and comprising between about 0.05 and 20 mg of a biodegradable elastomer per mm² of the surface, the biodegradable elastomer having a molecular weight of 75,000-240,000 kDa, and being present in an amount between 1 and 20 times the weight of the therapeutic agent in the first layer.

19. The coated implantable medical device of claim 18, where the hydrophobic therapeutic agent is a taxane therapeutic agent and the biodegradable elastomer is poly(lactic acid).

20. The coated implantable medical device of claim 18, wherein
a. the medical device is a radially-expandable vascular stent having an abluminal side and a luminal side defining a substantially cylindrical lumen and being movable from a radially expanded configuration having a diameter of about 2-10 mm, and a radially compressed configuration having a diameter of about 1-2.5 mm;
b. the coating is present on the abluminal surface, but not the luminal side of the vascular stent.
c. the first layer consists essentially of 0.05-0.90 μg of a paclitaxel therapeutic agent per mm² of the coated abluminal surface,
d. the second layer consists essentially of 0.05-18.00 μg of a biodegradable poly(D,L-lactic acid) elastomer having a molecular weight of 75,000-240,000 kDa per mm² of the coated abluminal surface,
e. the ratio of weight of the paclitaxel in the first layer to the poly(D,L- lactic acid)the second layer to the first layer is between about 1:1 and 1:20,
f. the coating having a durability characterized by a weight loss of less than 5% of the coating weight after crimping the medical device comprising the coating from the radially expanded configuration to the radially compressed configuration.
